(19) United States
(12) Patent Application Publication
(10) Pub. No.: US 2016/0106743 A1
(43) Pub. Date: Apr. 21, 2016

(54) TOPOISOMERASE II POISONS AND METHODS OF MAKING AND USING SAME

(71) Applicant: David Goldfarb, Victor, NY (US)

(72) Inventor: David Goldfarb, Victor, NY (US)

(73) Assignee: THE UNIVERSITY OF ROCHESTER, Rochester, NY (US)

(21) Appl. No.: 14/885,224

(22) Filed: Oct. 16, 2015

Related U.S. Application Data
(60) Provisional application No. 62/064,662, filed on Oct. 16, 2014.

(51) Int. Cl. A61K 31/4985; A61K 31/704; A61K 45/06

(52) U.S. Cl. CPC A61K 31/4985; A61K 45/06; A61K 31/704

(57) ABSTRACT

Disclosed herein are compositions of Formula I and an optional anticancer drug. Also disclosed herein are methods of treating or preventing cancer using the same.

### CROSS REFERENCE TO RELATED APPLICATIONS

This application claims the benefit of priority to U.S. Provisional Application 62/064,662, filed Oct. 16, 2014, which is incorporated by reference herein in its entirety.

### STATEMENT REGARDING GOVERNMENT SUPPORT

This invention was made with government support under Grant No. 1 R03 MH076395-01 awarded by the National Institutes of Health. The government has certain rights in this invention.

### FIELD

The subject matter disclosed herein relates generally to cancer therapy and to anticancer compounds. More specifically, the subject matter disclosed herein relates to compositions comprising topoisomerase II poisons and their use in the treatment of cancer.

### BACKGROUND

Mammalian Type II Topoisomerases (Top2α and Top2β) are targets of widely prescribed anticancer drugs; for example, worldwide sales of doxorubicin and Doxil, a lipid encapsulated formulation of doxorubicin, were recently about US$600 million. Top2s catalyze the untangling of concatenated DNA and relieve supercoiling that accumulates during DNA replication and transcription. Most anticancer drugs that target Top2 are poisons. In contrast to inhibitors, which inhibit the enzymes' ATPase activity, Top2 poisons lead to the accumulation of covalent catalytic intermediates containing double strand breaks (Top2 covalent complexes, Top2cc). Top2 poisons act either by inducing the formation of Topcc (e.g., ellipticine) or by preventing their religation (e.g., doxorubicin).

Doxorubicin is one of the most effective agents against breast cancer and numerous other solid and liquid tumors as well as non-cancerous conditions. The clinical use of doxorubicin is limited by cardiotoxicity and other dose-dependent toxic side-effects. The mechanism(s) of cardiotoxicity are poorly understood. To avoid cardiotoxicity and congestive heart failure patients are usually limited to a lifetime cumulative dose of 500 mg/m². The use of doxorubicin is especially limited in patients with preexisting heart conditions. No single agent can substitute for doxorubicin. Thus, a need exists for new drugs, formulations or compositions with improved anticancer efficacy, reduced side effects, or both. One approach is to seek enhancers that increase potency without a commensurate increase in side effects.

### SUMMARY

In accordance with the purposes of the disclosed materials and methods, as embodied and broadly described herein, the disclosed subject matter, in one aspect, relates to compounds, compositions and methods of making and using compounds and compositions. In specific aspects, the disclosed subject matter relates to cancer therapy and to anticancer compounds. More specifically, the subject matter disclosed herein relates to inhibitors and poisons of topoisomerase II and their use in the treatment of cancer.

Disclosed herein are compositions comprising (a) a topoisomerase II poisoning compound of Formula I:

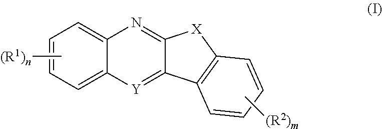

wherein
each R¹ is, independently, F, Cl, Br, I, OH, SH, (CH₂)ₖCO₂R⁵, (CH₂)ₖOC(O)R⁵, (CH₂)ₖNR⁵R⁶, or CN; or C₁₋₈ alkyl or cycloalkyl optionally substituted with one or more F, Cl, Br, I, OH, SH, ═O, ═S, NH₂, (CH₂)ₖCO₂R⁵, (CH₂)ₖNR⁵R⁶, OC₁₋₈ alkyl, or CN;
n is 0, 1, 2, 3, or 4;
each R² is, independently, F, Cl, Br, I, OH, SH, (CH₂)ₖCO₂R⁵, or (CH₂)ₖOC(O)R⁵, (CH₂)ₖNR⁵R⁶, or CN; or C₁₋₈ alkyl or cycloalkyl optionally substituted with one or more F, Cl, Br, I, OH, SH, ═O, ═S, NH₂, (CH₂)ₖCO₂R⁵, (CH₂)ₖNR⁵R⁶, OC₁₋₈ alkyl, or CN;
m is 0, 1, 2, 3, or 4;
X is N, NH, NC₁₋₈ alkyl, NC(O)R⁵, CH₂, CH—C₁₋₈ alkyl, C(C₁₋₈ alkyl)₂, CO, O, S, SO, or SO₂;
Y is N, NH, or CH;
R⁵ and R⁶ are, independently, H, C₁₋₈ alkyl, OC₁₋₈ alkyl, or cycloalkyl;
k is 0, 1, 2, 3, or 4;
a (+), (−), or (±) isomer, or a pharmaceutically acceptable salt or prodrug thereof; and

optionally (b) an anticancer drug, a (+), (−), or (±) isomer, or pharmaceutically acceptable salt or prodrug thereof. In some embodiments, Y is N. In some embodiments, X is NH or CO. In some embodiments, n is 0. In some embodiments, m is 0.

Also disclosed are compositions comprising (a) 6H-indolo[2,3-b]quinoxaline (Compound 1), and (b) an additional topoisomerase II inhibiting or poisoning compound. Also disclosed are compositions comprising (a) 11H-indeno[1,2-b]quinoxalin-11-one, and (b) an additional topoisomerase II inhibiting or poisoning compound. Also disclosed herein are compositions comprising (a) 6H-indolo[2,3-b]quinoxaline, and (b) doxorubicin, doxil, or myocet. Also disclosed herein are compositions comprising (a) 11H-indeno[1,2-b]quinoxalin-11-one, and (b) doxorubicin, doxil, or myocet.

In some embodiments, (b) comprises a topoisomerase II inhibitor or poison comprising a polycyclic chromophore, a synthetic intercalating drug, an antitumor antibiotic, a fluoroquinolone, a combination thereof, a (+), (−), or (±) isomer thereof, or a pharmaceutically acceptable salt or prodrug thereof. The polycyclic chromophore can comprise a benzoisoquinolinedione, an anthrapyrazole, a phenazine-1-carboxamide, a combination thereof, a (+), (−), or (±) isomer thereof, or a pharmaceutically acceptable salt or prodrug thereof. The synthetic intercalating drug can comprise an aminoacridine, a (+), (−), or (±) isomer thereof, or a pharmaceutically acceptable salt or prodrug thereof. The antitumor antibiotic can comprise an anthracyline, a (+), (−), or (±) isomer thereof, or a pharmaceutically acceptable salt or prodrug thereof. In some embodiments, (b) comprises aclarubicin, actinomycin, amsacrine, bleomycin, camptothecin, ciprofloxacin, dactinomycin, daunorubicin, doxorubicin, ellipticine, epipodophyllotoxin, etoposide, genistein, idarubicin, losoxantrone, merbarone, mitonafide, mitoxanthrone, paclitaxel, plicamycin, phosphate, teniposide, a combination thereof, a (+), (−), or (±) isomer thereof, or a pharmaceutically acceptable salt or prodrug thereof.

In some embodiments, (b) is a compound that inhibits and/or poisons topoisomerase I and II. The compound can comprise acridine-4-carboxamide N-[2-(dimethylamino)ethyl]acridine-4carboxamide, imidazoacridanone, a combination thereof, a (+), (−), or (±) isomer thereof, or a pharmaceutically acceptable salt or prodrug thereof.

Also disclosed herein are methods of treating or preventing cancer. For instance, disclosed herein is a method of treating or preventing cancer in a subject comprising administering to the subject (a) and (b) as disclosed herein. For instance, disclosed herein is a method of treating cancer comprising administering to a subject a pharmaceutically effective amount of (a) 6H-indolo[2,3-b]quinoxaline, and administering to a subject a pharmaceutically effective amount of (b) a topoisomerase II inhibiting or poisoning compound. For instance, disclosed herein is a method of treating cancer comprising administering to a subject a pharmaceutically effective amount of (a) 11H-indeno[1,2-b]quinoxalin-11-one, and administering to a subject a pharmaceutically effective amount of (b) a topoisomerase II inhibiting or poisoning compound. For instance, disclosed herein is a method of treating cancer comprising administering to a subject a pharmaceutically effective amount of (a) 6H-indolo[2,3-b]quinoxaline, and administering to a subject a pharmaceutically effective amount of (b) doxorubicin, doxil, or myocet. For instance, disclosed herein is a method of treating cancer comprising administering to a subject a pharmaceutically effective amount of (a) 11H-indeno[1,2-b]quinoxalin-11-one, and administering to a subject a pharmaceutically effective amount of (b) doxorubicin, doxil, or myocet.

Also disclosed are methods of treating or preventing cancer in a subject that has been administered a topoisomerase II poisoning compound of Formula I

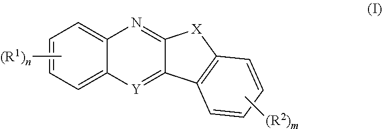

wherein
each R¹ is, independently, F, Cl, Br, I, OH, SH, (CH₂)ₖCO₂R⁵, (CH₂)ₖOC(O)R⁵, (CH₂)ₖNR⁵R⁶, or CN; or C₁₋₈ alkyl or cycloalkyl optionally substituted with one or more F, Cl, Br, I, OH, SH, ═O, ═S, NH₂, (CH₂)ₖCO₂R⁵, (CH₂)ₖNR⁵R⁶, OC₁₋₈ alkyl, or CN;
n is 0, 1, 2, 3, or 4;
each R² is, independently, F, Cl, Br, I, OH, SH, (CH₂)ₖCO₂R⁵, (CH₂)ₖOC(O)R⁵, (CH₂)ₖNR⁵R⁶, or CN; or C₁₋₈ alkyl or cycloalkyl optionally substituted with one or more F, Cl, Br, I, OH, SH, ═O, ═S, NH₂, (CH₂)ₖCO₂R⁵, (CH₂)ₖNR⁵R⁶, OC₁₋₈ alkyl, or CN;
m is 0, 1, 2, 3, or 4;
X is NH, NC₁₋₈ alkyl, NC(O)R⁵, CH₂, CH—C₁₋₈ alkyl, C(C₁₋₈ alkyl)₂, CO, O, S, SO, SO₂;
Y is N, CH;
R⁵ and R⁶ are, independently, H, C₁₋₈ alkyl, OC₁₋₈ alkyl, or cycloalkyl;
k is 0, 1, 2, 3, or 4;

(+), (−), and (±) isomers, and pharmaceutically acceptable salts and prodrugs thereof, comprising administering to a subject a pharmaceutically effective amount of (b) an anticancer drug.

Also disclosed is a method of treating or preventing cancer in a subject that has been administered (a) 6H-indolo[2,3-b]quinoxaline, comprising, in some embodiments, administering to the subject a pharmaceutically effective amount of (b) a topoisomerase II inhibiting or poisoning compound. Also disclosed is a method of treating or preventing cancer in a subject that has been administered (a) 11H-indeno[1,2-b]quinoxalin-11-one, comprising, in some embodiments, administering to the subject a pharmaceutically effective amount of (b) a topoisomerase II inhibiting or poisoning compound. Also disclosed is a method of treating or preventing cancer in a subject that has been administered (a) 6H-indolo[2,3-b]quinoxaline, comprising, in some embodiments, administering to the subject a pharmaceutically effective amount of (b) doxorubicin, doxil, or myocet. Also disclosed herein is a method of treating or preventing cancer in a subject that has been administered (a) 11H-indeno[1,2-b]quinoxalin-11-one, comprising, in some embodiments, administering to the subject a pharmaceutically effective amount of (b) doxorubicin, doxil, or myocet.

Also disclosed herein are methods of treating or preventing cancer in a subject that has been administered (b) an anticancer drug, comprising, in some embodiments, administering to the subject a pharmaceutically effective amount of (a) a topoisomerase II poisoning compound of Formula I

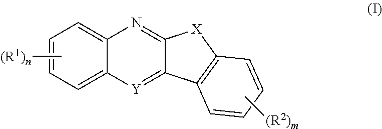

wherein
each R¹ is, independently, F, Cl, Br, I, OH, SH, (CH₂)ₖCO₂R⁵, (CH₂)ₖOC(O)R⁵, (CH₂)ₖNR⁵R⁶, or CN; or C₁₋₈ alkyl or cycloalkyl optionally substituted with one or more F, Cl, Br, I, OH, SH, ═O, ═S, NH₂, (CH₂)ₖCO₂R⁵, (CH₂)ₖNR⁵R⁶, OC₁₋₈ alkyl, or CN;
n is 0, 1, 2, 3, or 4;
each R² is, independently, F, Cl, Br, I, OH, SH, (CH₂)ₖCO₂R⁵, (CH₂)ₖOC(O)R⁵, (CH₂)ₖNR⁵R⁶, or CN; or C₁₋₈ alkyl or cycloalkyl optionally substituted with one or more F, Cl, Br, I, OH, SH, ═O, ═S, NH₂, (CH₂)ₖCO₂R⁵, (CH₂)ₖNR⁵R⁶, OC₁₋₈ alkyl, or CN;
m is 0, 1, 2, 3, or 4;
X is NH, NC₁₋₈ alkyl, NC(O)R⁵, CH₂, CH—C₁₋₈ alkyl, C(C₁₋₈ alkyl)₂, CO, O, S, SO, SO₂;
Y is N, CH;
R⁵ and R⁶ are, independently, H, C₁₋₈ alkyl, OC₁₋₈ alkyl, or cycloalkyl;
k is 0, 1, 2, 3, or 4;
(+), (−), and (±) isomers, and pharmaceutically acceptable salts and prodrugs thereof.

Also disclosed are methods of treating or preventing cancer in a subject that has been administered (b) a topoisomerase II inhibiting or poisoning compound, comprising, for instance, administering to the subject a pharmaceutically effective amount of (a) 6H-indolo[2,3-b]quinoxaline. Also disclosed are methods of treating or preventing cancer in a subject that has been administered (b) a topoisomerase II inhibiting or poisoning compound, comprising, for instance, administering to the subject a pharmaceutically effective amount of (a) 11H-indeno[1,2-b]quinoxalin-11-one. Also disclosed are methods of treating or preventing cancer in a subject that has been administered (b) doxorubicin, doxil, or myocet, comprising, for instance, administering to the subject a pharmaceutically effective amount of (a) 6H-indolo[2,3-b]quinoxaline. Also disclosed are methods of treating or preventing cancer in a subject that has been administered (b) doxorubicin, doxil, or myocet, comprising, for instance, administering to the subject a pharmaceutically effective amount of (a) 11H-indeno[1,2-b]quinoxalin-11-one.

Also disclosed are methods of potentiating (b) an anticancer drug in a subject, comprising, for instance, administering to the subject (a) a topoisomerase II poisoning compound of Formula I:

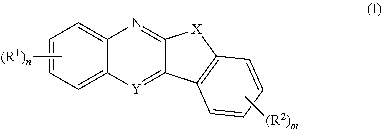

wherein
each R¹ is, independently, F, Cl, Br, I, OH, SH, (CH₂)ₖCO₂R⁵, (CH₂)ₖOC(O)R⁵, (CH₂)ₖNR⁵R⁶, or CN; or C₁₋₈ alkyl or cycloalkyl optionally substituted with one or more F, Cl, Br, I, OH, SH, ═O, ═S, NH₂, (CH₂)ₖCO₂R⁵, (CH₂)ₖNR⁵R⁶, OC₁₋₈ alkyl, or CN;
n is 0, 1, 2, 3, or 4;
each R² is, independently, F, Cl, Br, I, OH, SH, (CH₂)ₖCO₂R⁵, (CH₂)ₖOC(O)R⁵, (CH₂)ₖNR⁵R⁶, or CN; or C₁₋₈ alkyl or cycloalkyl optionally substituted with one or more F, Cl, Br, I, OH, SH, ═O, ═S, NH₂, (CH₂)ₖCO₂R⁵, (CH₂)ₖNR⁵R⁶, OC₁₋₈ alkyl, or CN;
m is 0, 1, 2, 3, or 4;
X is NH, NC₁₋₈ alkyl, NC(O)R⁵, CH₂, CH—C₁₋₈ alkyl, C(C₁₋₈ alkyl)₂, CO, O, S, SO, SO₂;
Y is N, CH;
R⁵ and R⁶ are, independently, H, C₁₋₈ alkyl, OC₁₋₈ alkyl, or cycloalkyl;
k is 0, 1, 2, 3, or 4;
(+), (−), and (±) isomers, and pharmaceutically acceptable salts and prodrugs thereof;

wherein (a) is administered in an amount effective to enhance the therapeutic efficacy of (b) the anticancer drug.

In some embodiments, (a) and (b) are administered simultaneously. In some embodiments, (a) and (b) are administered sequentially. In some embodiments, (a) and (b) are administered in a weight ratio of (a) to (b) from 1 to 1 to from 1 to 10. In some embodiments, (b) is administered in an amount of from 5% to 50% lower than an equivalent pharmaceutically effective amount administered without (a). In some embodiments, (b) is administered in an amount of from 5% to 50% lower than the recommended dose of 60 mg/m².

In some embodiments, the method further comprises administering (c) a third compound or composition, wherein the third compound or composition includes an additional anti-cancer drug. In some embodiments, the cancer is prostate cancer, lung cancer, breast cancer, brain cancer, ovarian cancer, lymphoma, leukemia, head and neck cancer, pancreatic cancer, cervical cancer, colon cancer, rectal cancer, endrometrial cancer, esophageal cancer, liver cancer, penile cancer, melanoma skin cancer, non-melanoma skin cancer, stomach cancer, testicular cancer, vaginal cancer, uterine cancer, vulvar cancer, paranasal cancer, oropharyngeal cancer, laryngeal cancer, or a combination thereof

Additional advantages will be set forth in part in the description that follows, and in part will be obvious from the description, or may be learned by practice of the aspects described below. The advantages described below will be realized and attained by means of the elements and combinations particularly pointed out in the appended claims. It is to be understood that both the foregoing general description and the following detailed description are exemplary and explanatory only and are not restrictive.

### BRIEF DESCRIPTION OF THE FIGURES

The accompanying Figures, which are incorporated in and constitute a part of this specification, illustrate several aspects described below.

FIG. 1 is a graph showing that the decatenation of kinetoplastid DNA by human Top2-α is inhibited by increasing concentrations of Compound 1.

FIG. 2 is a photograph of an electrophoresis gel showing that Compound 1 does not intercalate into DNA. The supercoiled (*) and relaxed (**) forms of plasmid DNA were used as substrates. Lane 1 is the control, lane 2 is in the presence of Topoisomerase 1 (Top1), lanes 3-5 are in the presence of doxorubicin plus Top1, lane 4 is in the presence of etoposide plus Top1, and lanes 8-9 are in the presence of Compound 1 plus Top1.

FIG. 3 is a pair of photographs of yeast cell dilutions from yeast expressing either overexpressing TOP1, TOP2, or TOPS. Media containing DMSO vehicle control is pictured on top and media containing Compound 1 is on the bottom. Compound 1 is toxic to cells overexpressing TOP2.

FIG. 4 is a graph showing nonlethal growth rate effects of Compound 1 in yeast strains expressing native (empty) or overexpressed levels of TOP2 (TOP2). Cells expressing native levels of TOP2 continue to grow at good rates even at high (100 μM) concentrations of Compound 1.

FIG. 5 is a photograph of a DNA gel demonstrating that Compound 1, like the well characterized Top2 poison etoposide, induces purified yeast Top2 to form stable double strand breaks (“lin” form) in supercoiled (“sc” form) plasmid DNA. Appearance of the lin form is consistent with the formation of Top2cc adducts.

FIGS. 6A-6C are graphs of cell viability of HT1080 cancer cells in media containing DMSO vehicle control (FIG. 6A), doxorubicin (FIG. 6B), and vinblastine (FIG. 6C) with various concentrations of Compound 1. FIG. 6D is a graph of cell viability at various concentrations of doxorubicin and Compound 1. The toxicity of doxorubicin, but not vinblastine, is enhanced in the presence of increasing concentrations of Compound 1.

FIG. 7 is a graph of cell viability of noncancerous MJT cells at various concentrations of doxorubicin with and without Compound 1. Compound 1 is neither toxic to noncancer cells nor does it enhance the toxicity of doxorubicin.

### DETAILED DESCRIPTION

The materials, compounds, compositions, articles, and methods described herein may be understood more readily by reference to the following detailed description of specific aspects of the disclosed subject matter and the Examples and Figures included therein.

Before the present materials, compounds, compositions, kits, and methods are disclosed and described, it is to be understood that the aspects described below are not limited to specific synthetic methods or specific reagents, as such may, of course, vary. It is also to be understood that the terminology used herein is for the purpose of describing particular aspects only and is not intended to be limiting.

### GENERAL DEFINITIONS

In this specification and in the claims that follow, reference will be made to a number of terms, which shall be defined to have the following meanings:

Throughout the description and claims of this specification the word “comprise” and other forms of the word, such as “comprising” and “comprises,” means including but not limited to, and is not intended to exclude, for example, other additives, components, integers, or steps.

As used in the description and the appended claims, the singular forms “a,” “an,” and “the” include plural referents unless the context clearly dictates otherwise. Thus, for example, reference to “a composition” includes mixtures of two or more such compositions; reference to “the compound” includes mixtures of two or more such compounds, and the like.

“Optional” or “optionally” means that the subsequently described event or circumstance can or cannot occur, and that the description includes instances where the event or circumstance occurs and instances where it does not.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed, then “less than or equal to” the value, “greater than or equal to the value,” and possible ranges between values are also disclosed, as appropriately understood by the skilled artisan.

As used herein, by a “subject” is meant an individual. Thus, the “subject” can include domesticated animals (e.g., cats, dogs, etc.), livestock (e.g., cattle, horses, pigs, sheep, goats, etc.), laboratory animals (e.g., mouse, rabbit, rat, guinea pig, etc.), and birds. “Subject” can also include a mammal, such as a primate or a human.

By “reduce” or other forms of the word, such as “reducing” or “reduction,” is meant lowering of an event or characteristic (e.g., bacterial growth). It is understood that this is typically in relation to some standard or expected value, in other words it is relative, but that it is not always necessary for the standard or relative value to be referred to. For example, “reduces cancer” means reducing the spread of a cancer relative to a standard or a control.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed.

By “treat” or other forms of the word, such as “treated” or “treatment,” is meant to administer a composition or to perform a method to reduce, prevent, or eliminate a particular characteristic or event (e.g., tumor growth, cancer). The term “control” is used synonymously with the term “treat.”

By “potentiate” is meant increase or enhance the effect of a therapeutic. For example, the therapeutic can be used at lower concentrations or can be administered at longer intervals than would occur in the absence of potentiating.

The term “anticancer” refers to the ability to treat or control cellular proliferation and/or tumor growth.

The term “therapeutically effective” means the amount of the composition used is of sufficient quantity to ameliorate one or more causes or symptoms of a disease or disorder. Such amelioration only requires a reduction or alteration, not necessarily elimination.

The term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problems or complications commensurate with a reasonable benefit/risk ratio.

The term “carrier” means a compound, composition, substance, or structure that, when in combination with a compound or composition, aids or facilitates preparation, storage, administration, delivery, effectiveness, selectivity, or any other feature of the compound or composition for its intended use or purpose. For example, a carrier can be selected to minimize any degradation of the active ingredient and to minimize any adverse side effects in the subject.

Unless stated to the contrary, a formula with chemical bonds shown only as solid lines and not as wedges or dashed lines contemplates each possible isomer, e.g., each enantiomer, diastereomer, and meso compound, and a mixture of isomers, such as a racemic or scalemic mixture.

It is understood that throughout this specification the identifiers “first” and “second” and the like are used solely to aid in distinguishing the various components and steps of the disclosed subject matter. The identifiers “first” and “second” and the like are not intended to imply any particular order, amount, preference, or importance to the components or steps modified by these terms.

### CHEMICAL DEFINITIONS

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, the heteroatoms, such as nitrogen, can have hydrogen substituents and/or any permissible substituents of organic compounds described herein which satisfy the valencies of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc.

“Z¹,” “Z²,” “Z³,” and “Z⁴” are used herein as generic symbols to represent various specific substituents. These symbols can be any substituent, not limited to those disclosed herein, and when they are defined to be certain substituents in one instance, they can, in another instance, be defined as some other substituents.

The term “aliphatic” as used herein refers to a non-aromatic hydrocarbon group and includes branched and unbranched, alkyl, alkenyl, or alkynyl groups.

The term “alkyl” as used herein is a branched or unbranched saturated hydrocarbon group of 1 to 24 carbon atoms, such as methyl, ethyl, n-propyl, isopropyl, n-butyl, isobutyl, t-butyl, pentyl, hexyl, heptyl, octyl, nonyl, decyl, dodecyl, tetradecyl, hexadecyl, eicosyl, tetracosyl, and the like. The alkyl group can also be substituted or unsubstituted. The alkyl group can be substituted with one or more groups including, but not limited to, alkyl, halogenated alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol, as described below.

Throughout the specification “alkyl” is generally used to refer to both unsubstituted alkyl groups and substituted alkyl groups; however, substituted alkyl groups are also specifically referred to herein by identifying the specific substituent(s) on the alkyl group. For example, the term “halogenated alkyl” specifically refers to an alkyl group that is substituted with one or more halide, e.g., fluorine, chlorine, bromine, or iodine. The term “alkoxyalkyl” specifically refers to an alkyl group that is substituted with one or more alkoxy groups, as described below. The term “alkylamino” specifically refers to an alkyl group that is substituted with one or more amino groups, as described below, and the like. When “alkyl” is used in one instance and a specific term such as “alkylalcohol” is used in another, it is not meant to imply that the term “alkyl” does not also refer to specific terms such as “alkylalcohol” and the like.

This practice is also used for other groups described herein. That is, while a term such as “cycloalkyl” refers to both unsubstituted and substituted cycloalkyl moieties, the substituted moieties can, in addition, be specifically identified herein; for example, a particular substituted cycloalkyl can be referred to as, e.g., an “alkylcycloalkyl.” Similarly, a substituted alkoxy can be specifically referred to as, e.g., a “halogenated alkoxy,” a particular substituted alkenyl can be, e.g., an “alkenylalcohol,” and the like. Again, the practice of using a general term, such as “cycloalkyl,” and a specific term, such as “alkylcycloalkyl,” is not meant to imply that the general term does not also include the specific term.

The term “alkoxy” as used herein is an alkyl group bound through a single, terminal ether linkage; that is, an “alkoxy” group can be defined as —OZ¹ where Z¹ is alkyl as defined above.

The term “alkenyl” as used herein is a hydrocarbon group of from 2 to 24 carbon atoms with a structural formula containing at least one carbon-carbon double bond. Asymmetric structures such as (Z¹Z²)C═C(Z³Z⁴) are intended to include both the E and Z isomers. This can be presumed in structural formulae herein wherein an asymmetric alkene is present, or it can be explicitly indicated by the bond symbol C═C. The alkenyl group can be substituted with one or more groups including, but not limited to, alkyl, halogenated alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol, as described below.

The term “alkynyl” as used herein is a hydrocarbon group of 2 to 24 carbon atoms with a structural formula containing at least one carbon-carbon triple bond. The alkynyl group can be substituted with one or more groups including, but not limited to, alkyl, halogenated alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol, as described below.

The term “aryl” as used herein is a group that contains any carbon-based aromatic group including, but not limited to, benzene, naphthalene, phenyl, biphenyl, phenoxybenzene, and the like. The term “heteroaryl” is defined as a group that contains an aromatic group that has at least one heteroatom incorporated within the ring of the aromatic group. Examples of heteroatoms include, but are not limited to, nitrogen, oxygen, sulfur, and phosphorus. The term “non-heteroaryl,” which is included in the term “aryl,” defines a group that contains an aromatic group that does not contain a heteroatom. The aryl or heteroaryl group can be substituted or unsubstituted. The aryl or heteroaryl group can be substituted with one or more groups including, but not limited to, alkyl, halogenated alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol as described herein. The term “biaryl” is a specific type of aryl group and is included in the definition of aryl. Biaryl refers to two aryl groups that are bound together via a fused ring structure, as in naphthalene, or are attached via one or more carbon-carbon bonds, as in biphenyl.

The term “cycloalkyl” as used herein is a non-aromatic carbon-based ring composed of at least three carbon atoms. Examples of cycloalkyl groups include, but are not limited to, cyclopropyl, cyclobutyl, cyclopentyl, cyclohexyl, etc. The term “heterocycloalkyl” is a cycloalkyl group as defined above where at least one of the carbon atoms of the ring is substituted with a heteroatom such as, but not limited to, nitrogen, oxygen, sulfur, or phosphorus. The cycloalkyl group and heterocycloalkyl group can be substituted or unsubstituted. The cycloalkyl group and heterocycloalkyl group can be substituted with one or more groups including, but not limited to, alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol as described herein.

The term “cycloalkenyl” as used herein is a non-aromatic carbon-based ring composed of at least three carbon atoms and containing at least one double bound, i.e., C═C. Examples of cycloalkenyl groups include, but are not limited to, cyclopropenyl, cyclobutenyl, cyclopentenyl, cyclopentadienyl, cyclohexenyl, cyclohexadienyl, and the like. The term “heterocycloalkenyl” is a type of cycloalkenyl group as defined above, and is included within the meaning of the term “cycloalkenyl,” where at least one of the carbon atoms of the ring is substituted with a heteroatom such as, but not limited to, nitrogen, oxygen, sulfur, or phosphorus. The cycloalkenyl group and heterocycloalkenyl group can be substituted or unsubstituted. The cycloalkenyl group and heterocycloalkenyl group can be substituted with one or more groups including, but not limited to, alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol as described herein.

The term “cyclic group” is used herein to refer to either aryl groups, non-aryl groups (i.e., cycloalkyl, heterocycloalkyl, cycloalkenyl, and heterocycloalkenyl groups), or both. Cyclic groups have one or more ring systems that can be substituted or unsubstituted. A cyclic group can contain one or more aryl groups, one or more non-aryl groups, or one or more aryl groups and one or more non-aryl groups.

The term “aldehyde” as used herein is represented by the formula —C(O)H. Throughout this specification “C(O)” or “CO” is a short hand notation for C═O.

The terms “amine” or “amino” as used herein are represented by the formula —NZ¹Z², where Z¹ and Z² can each be substitution group as described herein, such as hydrogen, an alkyl, halogenated alkyl, alkenyl, alkynyl, aryl, heteroaryl, cycloalkyl, cycloalkenyl, heterocycloalkyl, or heterocycloalkenyl group described above.

The term “carboxylic acid” as used herein is represented by the formula —C(O)OH. A “carboxylate” or “carboxyl” group as used herein is represented by the formula —C(O)O⁻.

The term “ester” as used herein is represented by the formula —OC(O)Z¹ or —C(O)OZ¹, where Z¹ can be an alkyl, halogenated alkyl, alkenyl, alkynyl, aryl, heteroaryl, cycloalkyl, cycloalkenyl, heterocycloalkyl, or heterocycloalkenyl group described above.

The term “ether” as used herein is represented by the formula Z¹OZ², where Z¹ and Z² can be, independently, an alkyl, halogenated alkyl, alkenyl, alkynyl, aryl, heteroaryl, cycloalkyl, cycloalkenyl, heterocycloalkyl, or heterocycloalkenyl group described above.

The term “ketone” as used herein is represented by the formula Z¹C(O)Z², where Z¹ and Z² can be, independently, an alkyl, halogenated alkyl, alkenyl, alkynyl, aryl, heteroaryl, cycloalkyl, cycloalkenyl, heterocycloalkyl, or heterocycloalkenyl group described above.

The term “halide” or “halogen” as used herein refers to the fluorine, chlorine, bromine, and iodine.

The term “hydroxyl” as used herein is represented by the formula —OH.

The term “nitro” as used herein is represented by the formula —NO₂.

The term “silyl” as used herein is represented by the formula —SiZ¹Z²Z³, where Z¹, Z², and Z³ can be, independently, hydrogen, alkyl, halogenated alkyl, alkoxy, alkenyl, alkynyl, aryl, heteroaryl, cycloalkyl, cycloalkenyl, heterocycloalkyl, or heterocycloalkenyl group described above.

The term “sulfonyl” is used herein to refer to the sulfo-oxo group represented by the formula —S(O)₂Z¹, where Z¹ can be hydrogen, an alkyl, halogenated alkyl, alkenyl, alkynyl, aryl, heteroaryl, cycloalkyl, cycloalkenyl, heterocycloalkyl, or heterocycloalkenyl group described above.

The term “sulfonylamino” or “sulfonamide” as used herein is represented by the formula —S(O)₂NH—.

The term “phosphonyl” is used herein to refer to the phospho-oxo group represented by the formula —P(O)(OZ¹)₂, where Z¹ can be hydrogen, an alkyl, halogenated alkyl, alkenyl, alkynyl, aryl, heteroaryl, cycloalkyl, cycloalkenyl, heterocycloalkyl, or heterocycloalkenyl group described above.

The term “thiol” as used herein is represented by the formula —SH.

The term “thio” as used herein is represented by the formula —S—.

“R¹,” “R²,” “R³,” “Rⁿ,” etc., where n is some integer, as used herein can, independently, possess one or more of the groups listed above. For example, if R¹ is a straight chain alkyl group, one of the hydrogen atoms of the alkyl group can optionally be substituted with a hydroxyl group, an alkoxy group, an amine group, an alkyl group, a halide, and the like. Depending upon the groups that are selected, a first group can be incorporated within second group or, alternatively, the first group can be pendant (i.e., attached) to the second group. For example, with the phrase “an alkyl group comprising an amino group,” the amino group can be incorporated within the backbone of the alkyl group. Alternatively, the amino group can be attached to the backbone of the alkyl group. The nature of the group(s) that is (are) selected will determine if the first group is embedded or attached to the second group.

Unless stated to the contrary, a formula with chemical bonds shown only as solid lines and not as wedges or dashed lines contemplates each possible isomer, e.g., each enantiomer, diastereomer, and meso compound, and a mixture of isomers, such as a racemic or scalemic mixture.

The following abbreviations are used herein: ADME, absorption, distribution, metabolism and excretion; DCM, dichloromethane; DPPP, bis(diphenylphosphino)propane; DIEA, diisopropylethylamine; DMF, dimethylformamide; DMSO, dimethyl sulfoxide; EtOAc, ethyl acetate; ESI, electrospray ionization; IBX, 2-iodoxybenzoic acid; KBr, potassium bromide; KF, potassium fluoride; MeCN, acetonitrile; MeOH, methyl alcohol; NCS, N-chlorosuccinimide; SAR, Structure Activity Relationship; TBAF, tetrabutylammonium fluoride; TBDMS, t-butyldimethylsilyl; TBDMSCl, t-butyldimethylsilyl chloride; Tf, trifluoromethanesulfonyl; THF, tetrahydrofuran.

A prodrug refers to a compound that is made more active in vivo. Certain compounds disclosed herein can also exist as prodrugs, as described in Hydrolysis in Drug and Prodrug Metabolism: Chemistry, Biochemistry, and Enzymology (Testa, Bernard and Mayer, Joachim M. Wiley-VHCA, Zurich, Switzerland 2003). Prodrugs of the compounds described herein are structurally modified forms of the compound that readily undergo chemical changes under physiological conditions to provide the compound. Additionally, prodrugs can be converted to the compound by chemical or biochemical methods in an ex vivo environment. For example, prodrugs can be slowly converted to a compound when placed in a transdermal patch reservoir with a suitable enzyme or chemical reagent. Prodrugs are often useful because, in some situations, they can be easier to administer than the compound, or parent drug. They can, for instance, be bioavailable by oral administration whereas the parent drug is not. The prodrug can also have improved solubility in pharmaceutical compositions over the parent drug. A wide variety of prodrug derivatives are known in the art, such as those that rely on hydrolytic cleavage or oxidative activation of the prodrug. An example, without limitation, of a prodrug would be a compound which is administered as an ester (the “prodrug”), but then is metabolically hydrolyzed to the carboxylic acid, the active entity. Additional examples include peptidyl derivatives of a compound.

Prodrugs of any of the disclosed compounds include, but are not limited to, carboxylate esters, carbonate esters, hemi-esters, phosphorus esters, nitro esters, sulfate esters, sulfoxides, amides, carbamates, azo compounds, phosphamides, glycosides, ethers, acetals, and ketals. Prodrug esters and carbonates can be formed, for example, by reacting one or more hydroxyl groups of compounds of Formula X with alkyl, alkoxy or aryl substituted acylating reagents using methods known to those of skill in the art to produce methyl carbonates, acetates, benzoates, pivalates and the like. Illustrative examples of prodrug esters of the compounds provided herein include, but are not limited to, compounds of Formula X having a carboxyl moiety wherein the free hydrogen is replaced by C₁-C₄ alkyl, C₁-C₇ alkanoyloxymethyl, 1-((C₁-C₅)alkanoyloxy)ethyl, 1-methyl-1-((C₁-C₅)alkanoyloxy)-ethyl, C₁-C₅ alkoxycarbonyloxymethyl, 1-((C₁-C₅)alkoxycarbonyloxy)ethyl, 1-methyl-1-((C₁-C₅)alkoxycarbonyloxy)ethyl, N—((C₁-C₅)alkoxycarbonyl)aminomethyl, 1-(N—((C₁-C₅)alkoxycarbonyl)amino)ethyl, 3-phthalidyl, 4-crotonolactonyl, gamma-butyrolacton-4-yl, di-N,N—(C₁-C₂)alkylamino(C₂-C₃)alkyl (e.g., beta-dimethylaminoethyl), carbamoyl-(C₁-C₂)alkyl, N,N-di(C₁-C₂)alkylcarbamoyl-(C₁-C₂)alkyl and piperidino-, pyrrolidino- or morpholino(C₂-C₃)alkyl. Oligopeptide modifications and biodegradable polymer derivatives (as described, for example, in Int. J. Pharm. 115, 61-67, 1995) are within the scope of the present disclosure. Methods for selecting and preparing suitable prodrugs are provided, for example, in the following: T. Higuchi and V. Stella, “Prodrugs as Novel Delivery Systems,” Vol. 14, ACS Symposium Series, 1975; H. Bundgaard, Design of Prodrugs, Elsevier, 1985; and Bioreversible Carriers in Drug Design, ed. Edward Roche, American Pharmaceutical Association and Pergamon Press, 1987

Reference will now be made in detail to specific aspects of the disclosed materials, compounds, compositions, articles, and methods, examples of which are illustrated in the accompanying Examples.

The examples below are intended to further illustrate certain aspects of the methods and compounds described herein, and are not intended to limit the scope of the claims.

### Compounds

Disclosed herein are, for instance, anti-cancer compositions comprising compound (a) and compound (b).

### Compound (a)

In some embodiments, the compositions disclosed herein comprise a compound (a) that can poison topoisomerase II and/or enhance the effect of a topoisomerase II inhibitor or poison. For instance, the compound (a) can be a compound of Formula I:

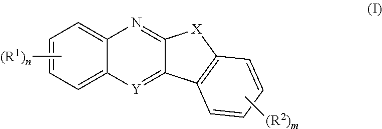

wherein
each R¹ is, independently, F, Cl, Br, I, OH, SH, (CH₂)ₖCO₂R⁵, (CH₂)ₖOC(O)R⁵, (CH₂)ₖNR⁵R⁶, or CN; or C₁₋₈ alkyl or cycloalkyl optionally substituted with one or more F, Cl, Br, I, OH, SH, ═O, ═S, NH₂, (CH₂)ₖCO₂R⁵, (CH₂)ₖNR⁵R⁶, OC₁₋₈ alkyl, or CN;
n is 0, 1, 2, 3, or 4;
each R² is, independently, F, Cl, Br, I, OH, SH, (CH₂)ₖCO₂R⁵, or (CH₂)ₖOC(O)R⁵, (CH₂)ₖNR⁵R⁶, or CN; or C₁₋₈ alkyl or cycloalkyl optionally substituted with one or more F, Cl, Br, I, OH, SH, ═O, ═S, NH₂, (CH₂)ₖCO₂R⁵, (CH₂)ₖNR⁵R⁶, OC₁₋₈ alkyl, or CN;
m is 0, 1, 2, 3, or 4;
X is NH, NC₁₋₈ alkyl, NC(O)R⁵, CH₂, CH—C₁₋₈ alkyl, C(C₁₋₈ alkyl)₂, CO, O, S, SO, or SO₂;
Y is N, or CH;
R⁵ and R⁶ are, independently, H, C₁₋₈ alkyl, OC₁₋₈ alkyl, or cycloalkyl;
k is 0, 1, 2, 3, or 4;
a (+), (−), or (±) isomer, or pharmaceutically acceptable salt or prodrug thereof

It is to be understood that Formula I encompasses various resonance structures, and can be equivalently displayed as follows in Formula I-a, wherein the dotted lines represent single or double bonds:

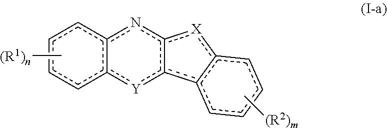

In a specific example, two resonance structures of Formula I where X is NH and Y is N can exist where X is N and Y is NH. There are of course other resonance structures, which are also contemplated herein. It should be understood that all references to a particular compound or group of compounds made herein by referring to Formula I, including references to various substituents in Formula (I), are meant to also contemplate a reference to each of the various resonance structures of Formula (I).

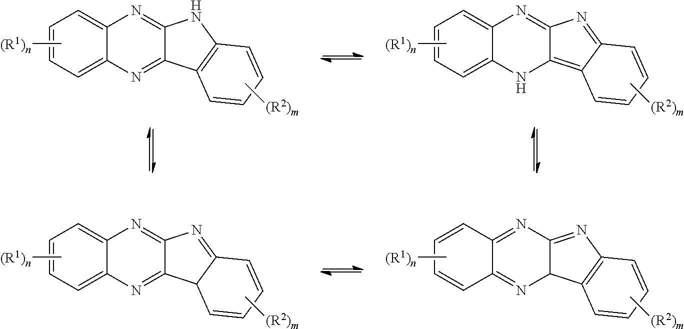

In some embodiments, Y is N, and the compound is of general Formula I-1, a (+), (−), or (±) isomer, or pharmaceutically acceptable salt or prodrug thereof:

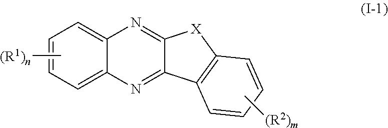

In one embodiment, X is CO, Y is N, n is 0, and m is 0, and the compound is 11H-indeno[1,2-b]quinoxalin-11-one, a (+), (−), or (±) isomer, or pharmaceutically acceptable salt or prodrug thereof, as shown in Formula I-2:

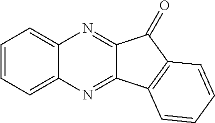 [SMILES: O=C1c2ccccc2-c2nc3ccccc3nc21]

In one embodiment, X is NH, Y is N, n is 0, and m is 0, and the compound is 6H-indolo[2,3-b]quinoxaline. It is to be understood that the resonance structures are contemplated of all compounds disclosed herein. For instance, the following resonance structures of Formula I-3 both fall within the scope of Formula I:

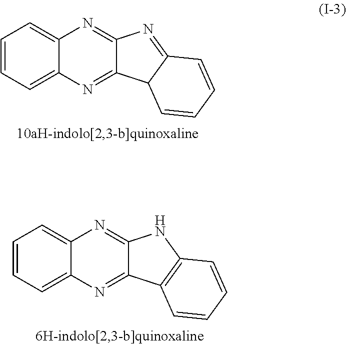 [SMILES: C1=CC2=Nc3nc4ccccc4nc3C2C=C1 ; c1ccc2nc3c(nc2c1)[nH]c1ccccc13]

Compound (a) can be synthesized in a variety of ways. For instance, compound (a) can be synthesized as shown below:

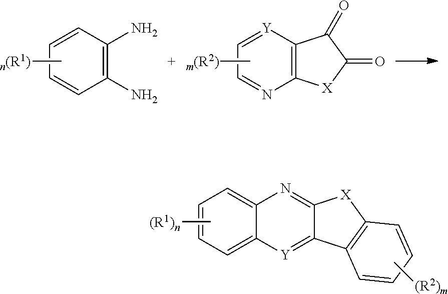

### Compound (b)

The compositions disclosed herein also comprise compound (b), an anticancer drug. Compound (b) can be an anticancer drug such as a topoisomerase I inhibitor or poison, a topoisomerase II inhibitor or poison, or a composition that inhibits or poisons topoisomerase I and II. Inhibitors of topoisomerase can be compounds that have inhibit the ATPase, whereas poisons of topoisomerase can be compounds that increase formation or stabilization of the topoisomerase covalent complex.

In some embodiments, compound (b) is a topoisomerase II inhibitor or poison. Topoisomerase II inhibitors can include synthetic intercalating drugs, such as the aminoacridines, antitumor antibiotics such as anthracylines, including doxorubicin, and plant-derived agents such as the ellipticines, epipodophyllotoxins, and camptothecins. In addition, some topoisomerase II inhibitors do not intercalate into the DNA, and these include etoposide, teniposide, and compounds such as merbarone and aclarubicine, which are not believed to form cleavable complexes with DNA.

In some embodiments, topoisomerase II inhibitors and poisons can include polycyclic chromophores bearing a flexible cationic side chains. Examples of polycyclic chromophores bearing flexible cationic side chains include, but are not limited to, benzoisoquinolinediones such as mitonafide, the anthrapyrazoles such as losoxantrone, and the phenazine-1-carboxamides.

In some embodiments, the topoisomerase II inhibitor or poison comprises aclarubicine, actinomycin, amsacrine, bleomycin, camptothecin, ciprofloxacin, dactinomycin, daunorubicin, doxorubicin, ellipticine, epipodophyllotoxin, etoposide, genistein, idarubicin, losoxantrone, merbarone, mitonafide, mitoxanthrone, paclitaxel, plicamycin, phosphate, teniposide, a combination thereof, a (+), (−), or (±) isomer thereof, or a pharmaceutically acceptable salt or prodrug thereof

Topoisomerase II inhibitors and poisons can have a wide variety of structures. In some embodiments, the topoisomerase II inhibitors or poisons comprise fused tetracyclic systems, for example, the ellipticines and batracyclin. In some embodiments, the topoisomerase II inhibitors or poisons comprise two 6-membered rings linked to a third 6-membered ring via a 5-membered ring.

Topoisomerase II inhibitors or poisons can also comprise methyl-substituted derivatives of benzopsoralen and benzoangelicin, such as 4-hydroxymethyl benzopsoralen. Topoisomerase II inhibitors or poisons can also comprise substituted tetracyclic fused quinoline derivatives.

Topoisomerase II inhibitors such as doxorubicin can be an effective anti-cancer drug for various solid and liquid tumors. The topoisomerase II inhibiting activity of doxorubicin is not the only mechanism that has been proposed to explain its therapeutic efficacy.

The potency of topoisomerase II inhibitors and poisons can increase with increased target concentration. Over-expressing topoisomerase II increases the potency of compounds (b) comprising topoisomerase II inhibitors or poisons. Without wishing to be bound to theory, it is believed that the compounds (a) disclosed herein can transiently stabilize topoisomerase II-DNA complexes, thereby increasing the effective target concentration of compounds (b).

In some embodiments, the anticancer drug comprises a mixed inhibitor or poison that inhibits topoisomerase I and II. Examples of such compounds include, but are not limited to, the acridine-4-carboxamide N-[2-(dimethylamino)ethyl]acridine-4carboxamide (DACA), the imidazoacridanone, and various tetracyclic chromophores. DACA has been found to have a different in vitro cytotoxicity profile to amsacrine and etoposide, and can be active against cell lines that show both P-glycoprotein-mediated (transport) drug resistance and “atypical” or “altered” multidrug resistance. Thus, in embodiments with a combination of drugs that act via a different cytotoxic mechanism, DACA and related compounds can circumvent multidrug resistance.

### Pharmaceutical Compositions

The compounds described herein or derivatives thereof can be provided in a pharmaceutical composition. Depending on the intended mode of administration, the pharmaceutical composition can be in the form of solid, semi-solid or liquid dosage forms, such as, for example, tablets, suppositories, pills, capsules, powders, liquids, or suspensions, e.g., in unit dosage form suitable for single administration of a precise dosage. The compositions can include a therapeutically effective amount of one or more of the compounds described herein or derivatives thereof in combination with a pharmaceutically acceptable carrier and, in addition, can include other medicinal agents, pharmaceutical agents, carriers, or diluents. By pharmaceutically acceptable is meant a material that is not biologically or otherwise undesirable, which can be administered to a subject along with the selected compound without causing unacceptable biological effects or interacting in a deleterious manner with the other components of the pharmaceutical composition in which it is contained.

As used herein, the term carrier encompasses any excipient, diluent, filler, salt, buffer, stabilizer, solubilizer, lipid, stabilizer, or other material well known in the art for use in pharmaceutical formulations. The choice of a carrier for use in a composition will depend upon the intended route of administration for the composition. The preparation of pharmaceutically acceptable carriers and formulations containing these materials is described in, e.g., REMINGTON'S PHARMACEUTICAL SCIENCES, 21st Edition, ed. University of the Sciences in Philadelphia, Lippincott, Williams & Wilkins, Philadelphia Pa., 2005. Examples of pharmaceutically acceptable carriers include buffers such as phosphate buffers, citrate buffer, and buffers with other organic acids; antioxidants including ascorbic acid; low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™ (ICI, Inc.; Bridgewater, N.J.), polyethylene glycol (PEG), and PLURONICS™ (BASF; Florham Park, N.J.).

Compositions containing one or more of the compounds described herein or derivatives thereof suitable for parenteral injection can comprise physiologically acceptable sterile aqueous or nonaqueous solutions, dispersions, suspensions or emulsions, and sterile powders for reconstitution into sterile injectable solutions or dispersions. Examples of suitable aqueous and nonaqueous carriers, diluents, solvents or vehicles include water, ethanol, polyols (propyleneglycol, polyethyleneglycol, glycerol, and the like), suitable mixtures thereof, vegetable oils (such as olive oil) and injectable organic esters such as ethyl oleate. Proper fluidity can be maintained, for example, by the use of a coating such as lecithin, by the maintenance of a desirable particle size in the case of dispersions and by the use of surfactants.

These compositions can also contain adjuvants such as preserving, wetting, emulsifying, and dispensing agents. Prevention of the action of microorganisms can be promoted by various antibacterial and antifungal agents, for example, parabens, chlorobutanol, phenol, sorbic acid, and the like. Isotonic agents, for example, sugars, sodium chloride, and the like can also be included. Prolonged absorption of the injectable pharmaceutical form can be brought about by the use of agents delaying absorption, for example, aluminum monostearate and gelatin.

Solid dosage forms for oral administration of the compounds described herein or derivatives thereof include capsules, tablets, pills, powders, and granules. In such solid dosage forms, the compounds described herein or derivatives thereof is admixed with at least one inert customary excipient (or carrier) such as sodium citrate or dicalcium phosphate or (a) fillers or extenders, as for example, starches, lactose, sucrose, glucose, mannitol, and silicic acid, (b) binders, as for example, carboxymethylcellulose, alignates, gelatin, polyvinylpyrrolidone, sucrose, and acacia, (c) humectants, as for example, glycerol, (d) disintegrating agents, as for example, agar-agar, calcium carbonate, potato or tapioca starch, alginic acid, certain complex silicates, and sodium carbonate, (e) solution retarders, as for example, paraffin, (f) absorption accelerators, as for example, quaternary ammonium compounds, (g) wetting agents, as for example, cetyl alcohol, and glycerol monostearate, (h) adsorbents, as for example, kaolin and bentonite, and (i) lubricants, as for example, talc, calcium stearate, magnesium stearate, solid polyethylene glycols, sodium lauryl sulfate, or mixtures thereof. In the case of capsules, tablets, and pills, the dosage forms can also comprise buffering agents.

Solid compositions of a similar type can also be employed as fillers in soft and hard-filled gelatin capsules using such excipients as lactose or milk sugar as well as high molecular weight polyethylene glycols, and the like.

Solid dosage forms such as tablets, dragees, capsules, pills, and granules can be prepared with coatings and shells, such as enteric coatings and others known in the art. They can contain opacifying agents and can also be of such composition that they release the active compound or compounds in a certain part of the intestinal tract in a delayed manner. Examples of embedding compositions that can be used are polymeric substances and waxes. The active compounds can also be in micro-encapsulated form, if appropriate, with one or more of the above-mentioned excipients.

Liquid dosage forms for oral administration of the compounds described herein or derivatives thereof include pharmaceutically acceptable emulsions, solutions, suspensions, syrups, and elixirs. In addition to the active compounds, the liquid dosage forms can contain inert diluents commonly used in the art, such as water or other solvents, solubilizing agents, and emulsifiers, as for example, ethyl alcohol, isopropyl alcohol, ethyl carbonate, ethyl acetate, benzyl alcohol, benzyl benzoate, propylene glycol, 1,3-butyleneglycol, dimethylformamide, oils, in particular, cottonseed oil, groundnut oil, corn germ oil, olive oil, castor oil, sesame oil, glycerol, tetrahydrofurfuryl alcohol, polyethylene glycols, and fatty acid esters of sorbitan, or mixtures of these substances, and the like.

Besides such inert diluents, the composition can also include additional agents, such as wetting, emulsifying, suspending, sweetening, flavoring, or perfuming agents.

Suspensions, in addition to the active compounds, can contain additional agents, as for example, ethoxylated isostearyl alcohols, polyoxyethylene sorbitol and sorbitan esters, microcrystalline cellulose, aluminum metahydroxide, bentonite, agar-agar and tragacanth, or mixtures of these substances, and the like.

Compositions of the compounds described herein or derivatives thereof for rectal administrations are optionally suppositories, which can be prepared by mixing the compounds with suitable non-irritating excipients or carriers such as cocoa butter, polyethyleneglycol or a suppository wax, which are solid at ordinary temperatures but liquid at body temperature and therefore, melt in the rectum or vaginal cavity and release the active component.

Dosage forms for topical administration of the compounds described herein or derivatives thereof include ointments, powders, sprays, and inhalants. The compounds described herein or derivatives thereof are admixed under sterile conditions with a physiologically acceptable carrier and any preservatives, buffers, or propellants as can be required. Ophthalmic formulations, ointments, powders, and solutions are also contemplated as being within the scope of the compositions.

The compositions can include one or more of the compounds described herein and a pharmaceutically acceptable carrier. As used herein, the term pharmaceutically acceptable salt refers to those salts of the compound described herein or derivatives thereof that are, within the scope of sound medical judgment, suitable for use in contact with the tissues of subjects without undue toxicity, irritation, allergic response, and the like, commensurate with a reasonable benefit/risk ratio, and effective for their intended use, as well as the zwitterionic forms, where possible, of the compounds described herein. The term salts refers to the relatively non-toxic, inorganic and organic acid addition salts of the compounds described herein. These salts can be prepared in situ during the isolation and purification of the compounds or by separately reacting the purified compound in its free base form with a suitable organic or inorganic acid and isolating the salt thus formed. Representative salts include the hydrobromide, hydrochloride, sulfate, bisulfate, nitrate, acetate, oxalate, valerate, oleate, palmitate, stearate, laurate, borate, benzoate, lactate, phosphate, tosylate, citrate, maleate, fumarate, succinate, tartrate, naphthylate mesylate, glucoheptonate, lactobionate, methane sulphonate, and laurylsulphonate salts, and the like. These can include cations based on the alkali and alkaline earth metals, such as sodium, lithium, potassium, calcium, magnesium, and the like, as well as non-toxic ammonium, quaternary ammonium, and amine cations including, but not limited to ammonium, tetramethylammonium, tetraethylammonium, methylamine, dimethylamine, trimethylamine, triethylamine, ethylamine, and the like. See S. M. Barge et al., J. PHARM. SCI. (1977) 66, 1, which is incorporated herein by reference in its entirety, at least for salts taught therein.

Administration of the compounds and compositions described herein or pharmaceutically acceptable salts thereof to a subject can be carried out using therapeutically effective amounts of the compounds and compositions described herein or pharmaceutically acceptable salts thereof as described herein for periods of time effective to treat a disorder.

The effective amount of the compounds and compositions described herein or pharmaceutically acceptable salts or prodrugs thereof as described herein can be determined by one of ordinary skill in the art and includes exemplary dosage amounts for a mammal of from about 0.5 mg/kg to about 200 mg/kg of body weight of active compound per day, which can be administered in a single dose or in the form of individual divided doses, such as from 1 to 4 times per day. Alternatively, the dosage amount can be from about 0.5 mg/kg to about 150 mg/kg of body weight of active compound per day, about 0.5 to 100 mg/kg of body weight of active compound per day, about 0.5 to about 75 mg/kg of body weight of active compound per day, about 0.5 to about 50 mg/kg of body weight of active compound per day, about 0.5 to about 25 mg/kg of body weight of active compound per day, about 1 to about 20 mg/kg of body weight of active compound per day, about 1 to about 10 mg/kg of body weight of active compound per day, about 20 mg/kg of body weight of active compound per day, about 10 mg/kg of body weight of active compound per day, or about 5 mg/kg of body weight of active compound per day. The expression effective amount, when used to describe an amount of compound in a method, refers to the amount of a compound that achieves the desired pharmacological effect or other effect, for example an amount that results in bacterial enzyme inhibition.

In some embodiments, compounds (a) and (b) are provided in the pharmaceutical composition in a weight ratio of (a) to (b) from 1 to 1 to from 1 to 10.

Those of skill in the art will understand that the specific dose level and frequency of dosage for any particular subject can be varied and will depend upon a variety of factors, including the activity of the specific compound employed, the metabolic stability and length of action of that compound, the species, age, body weight, general health, sex and diet of the subject, the mode and time of administration, rate of excretion, drug combination, and severity of the particular condition.

### Methods of Treating or Preventing Cancer

Disclosed herein are methods of treating or preventing cancer. For instance, disclosed herein is a method of treating or preventing cancer in a subject, the method comprising administering to the subject (a) and (b) as disclosed herein. Also disclosed herein is a method of treating cancer comprising administering to a subject a pharmaceutically effective amount of (a) a topoisomerase II poisoning compound of Formula I

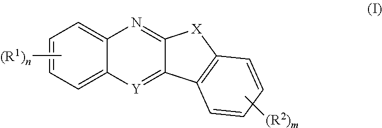

wherein
each R¹ is, independently, F, Cl, Br, I, OH, SH, (CH₂)ₖCO₂R⁵, (CH₂)ₖOC(O)R⁵, (CH₂)ₖNR⁵R⁶, or CN; or C₁₋₈ alkyl or cycloalkyl optionally substituted with one or more F, Cl, Br, I, OH, SH, ═O, ═S, NH₂, (CH₂)ₖCO₂R⁵, (CH₂)ₖNR⁵R⁶, OC₁₋₈ alkyl, or CN;
n is 0, 1, 2, 3, or 4;
each R² is, independently, F, Cl, Br, I, OH, SH, (CH₂)ₖCO₂R⁵, (CH₂)ₖOC(O)R⁵, (CH₂)ₖNR⁵R⁶, or CN; or C₁₋₈ alkyl or cycloalkyl optionally substituted with one or more F, Cl, Br, I, OH, SH, ═O, ═S, NH₂, (CH₂)ₖCO₂R⁵, (CH₂)ₖNR⁵R⁶, OC₁₋₈ alkyl, or CN;
m is 0, 1, 2, 3, or 4;
X is NH, NC₁₋₈ alkyl, NC(O)R⁵, CH₂, CH—C₁₋₈ alkyl, C(C₁₋₈ alkyl)₂, CO, O, S, SO, SO₂;
Y is N, CH;
R⁵ and R⁶ are, independently, H, C₁₋₈ alkyl, OC₁₋₈ alkyl, or cycloalkyl;
k is 0, 1, 2, 3, or 4;

a (+), (−), or (±) isomer thereof, or a pharmaceutically acceptable salt or prodrug thereof; and administering to a subject a pharmaceutically effective amount of (b) an anticancer drug.

For instance, disclosed herein is a method of treating cancer comprising administering to a subject a pharmaceutically effective amount of (a) 6H-indolo[2,3-b]quinoxaline, and administering to a subject a pharmaceutically effective amount of (b) a topoisomerase II inhibiting compound. For instance, disclosed herein is a method of treating cancer comprising administering to a subject a pharmaceutically effective amount of (a) 11H-indeno[1,2-b]quinoxalin-11-one, and administering to a subject a pharmaceutically effective amount of (b) a topoisomerase II inhibiting compound. For instance, disclosed herein is a method of treating cancer comprising administering to a subject a pharmaceutically effective amount of (a) 6H-indolo[2,3-b]quinoxaline, and administering to a subject a pharmaceutically effective amount of (b) doxorubicin. For instance, disclosed herein is a method of treating cancer comprising administering to a subject a pharmaceutically effective amount of (a) 11H-indeno[1,2-b]quinoxalin-11-one, and administering to a subject a pharmaceutically effective amount of (b) doxorubicin.

Also disclosed are methods of treating or preventing cancer in a subject that has been administered (a) a topoisomerase II poisoning compound of Formula I

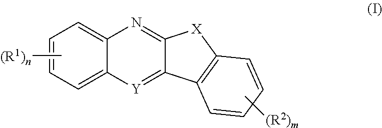

wherein
each R¹ is, independently, F, Cl, Br, I, OH, SH, (CH₂)ₖCO₂R⁵, (CH₂)ₖOC(O)R⁵, (CH₂)ₖNR⁵R⁶, or CN; or C₁₋₈ alkyl or cycloalkyl optionally substituted with one or more F, Cl, Br, I, OH, SH, ═O, ═S, NH₂, (CH₂)ₖCO₂R⁵, (CH₂)ₖNR⁵R⁶, OC₁₋₈ alkyl, or CN;
n is 0, 1, 2, 3, or 4;
each R² is, independently, F, Cl, Br, I, OH, SH, (CH₂)ₖCO₂R⁵, (CH₂)ₖOC(O)R⁵, (CH₂)ₖNR⁵R⁶, or CN; or C₁₋₈ alkyl or cycloalkyl optionally substituted with one or more F, Cl, Br, I, OH, SH, ═O, ═S, NH₂, (CH₂)ₖCO₂R⁵, (CH₂)ₖNR⁵R⁶, OC₁₋₈ alkyl, or CN;
m is 0, 1, 2, 3, or 4;
X is NH, NC₁₋₈ alkyl, NC(O)R⁵, CH₂, CH—C₁₋₈ alkyl, C(C₁₋₈ alkyl)₂, CO, O, S, SO, SO₂;
Y is N, CH;
R⁵ and R⁶ are, independently, H, C₁₋₈ alkyl, OC₁₋₈ alkyl, or cycloalkyl;
k is 0, 1, 2, 3, or 4;

(+), (−), and (±) isomers, and pharmaceutically acceptable salts and prodrugs thereof, and administering to a subject a pharmaceutically effective amount of (b) an anticancer drug. It is to be understood that Formula I encompasses various resonance structures, and can be equivalently displayed as follows in Formula I-a, wherein the dotted lines represent single or double bonds, as valence permits:

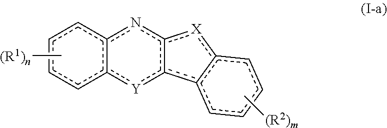

In some embodiments, Y is N, and the compound is of general Formula I-1, a (+), (−), or (±) isomer, or pharmaceutically acceptable salt or prodrug thereof:

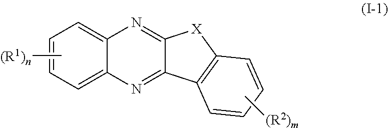

In one embodiment, X is CO, Y is N, n is 0, and m is 0, and the compound is 11H-indeno[1,2-b]quinoxalin-11-one, a (+), (−), or (±) isomer, or pharmaceutically acceptable salt or prodrug thereof, as shown in Formula I-2:

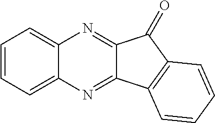 [SMILES: O=C1c2ccccc2-c2nc3ccccc3nc21]

In one embodiment, X is NH, Y is N, n is 0, and m is 0, and the compound is 6H-indolo[2,3-b]quinoxaline (referred to herein as Compound 1). It is to be understood that the resonance structures are contemplated of all compounds disclosed herein. For instance, the following resonance structures of Formula I-3 both fall within the scope of Formula I:

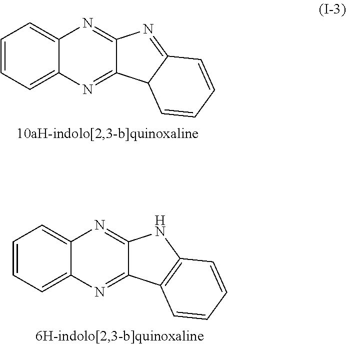 [SMILES: C1=CC2=Nc3nc4ccccc4nc3C2C=C1 ; c1ccc2nc3c(nc2c1)[nH]c1ccccc13]

Also disclosed is a method of treating or preventing cancer in a subject that has been administered (a) 6H-indolo[2,3-b]quinoxaline, the method comprising, in some embodiments, administering to the subject a pharmaceutically effective amount of (b) a topoisomerase II inhibiting or poisoning compound. Also disclosed is a method of treating or preventing cancer in a subject that has been administered (a) 11H-indeno[1,2-b]quinoxalin-11-one, comprising, in some embodiments, administering to the subject a pharmaceutically effective amount of (b) a topoisomerase II inhibiting or poisoning compound. Also disclosed is a method of treating or preventing cancer in a subject that has been administered (a) 6H-indolo[2,3-b]quinoxaline, comprising, in some embodiments, administering to the subject a pharmaceutically effective amount of (b) doxorubicin, doxil, or myocet. Also disclosed herein is a method of treating or preventing cancer in a subject that has been administered (a) 11H-indeno[1,2-b]quinoxalin-11-one, comprising, in some embodiments, administering to the subject a pharmaceutically effective amount of (b) doxorubicin, doxil, or myocet.

Also disclosed herein are methods of treating or preventing cancer in a subject that has been administered (b) an anticancer drug, the methods comprising, in some embodiments, administering to the subject a pharmaceutically effective amount of (a) a topoisomerase II poison of Formula I

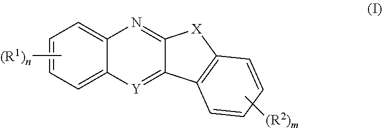

wherein
each R¹ is, independently, F, Cl, Br, I, OH, SH, (CH₂)ₖCO₂R⁵, (CH₂)ₖOC(O)R⁵, (CH₂)ₖNR⁵R⁶, or CN; or C₁₋₈ alkyl or cycloalkyl optionally substituted with one or more F, Cl, Br, I, OH, SH, ═O, ═S, NH₂, (CH₂)ₖCO₂R⁵, (CH₂)ₖNR⁵R⁶, OC₁₋₈ alkyl, or CN;
n is 0, 1, 2, 3, or 4;
each R² is, independently, F, Cl, Br, I, OH, SH, (CH₂)ₖCO₂R⁵, (CH₂)ₖOC(O)R⁵, (CH₂)ₖNR⁵R⁶, or CN; or C₁₋₈ alkyl or cycloalkyl optionally substituted with one or more F, Cl, Br, I, OH, SH, ═O, ═S, NH₂, (CH₂)ₖCO₂R⁵, (CH₂)ₖNR⁵R⁶, OC₁₋₈ alkyl, or CN;
m is 0, 1, 2, 3, or 4;
X is NH, NC₁₋₈ alkyl, NC(O)R⁵, CH₂, CH—C₁₋₈₈ alkyl, C(C₁₋₈ alkyl)₂, CO, O, S, SO, SO₂;
Y is N, CH;
R⁵ and R⁶ are, independently, H, C₁₋₈ alkyl, OC₁₋₈ alkyl, or cycloalkyl;
k is 0, 1, 2, 3, or 4;
   (+), (−), and (±) isomers, and pharmaceutically acceptable salts and prodrugs thereof. Also disclosed are methods of treating or preventing cancer in a subject that has been administered (b) a topoisomerase II inhibiting or poisoning compound, comprising, for instance, administering to the subject a pharmaceutically effective amount of (a) 6H-indolo[2,3-b]quinoxaline. Also disclosed are methods of treating or preventing cancer in a subject that has been administered (b) a topoisomerase II inhibiting or poisoning compound, comprising, for instance, administering to the subject a pharmaceutically effective amount of (a) 11H-indeno[1,2-b]quinoxalin-11-one. Also disclosed are methods of treating or preventing cancer in a subject that has been administered (b) doxorubicin, doxil, or myocet comprising, for instance, administering to the subject a pharmaceutically effective amount of (a) 6H-indolo[2,3-b]quinoxaline. Also disclosed are methods of treating or preventing cancer in a subject that has been administered (b) doxorubicin, doxil, or myocet comprising, for instance, administering to the subject a pharmaceutically effective amount of (a) 11H-indeno[1,2-b]quinoxalin-11-one.

Also disclosed are methods of potentiating (b) an anticancer drug in a subject, comprising, for instance, administering to the subject (a) a topoisomerase II poison of Formula I:

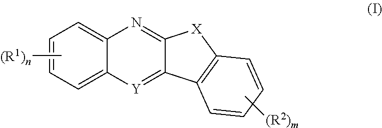

wherein

each R¹ is, independently, F, Cl, Br, I, OH, SH, (CH₂)ₖCO₂R⁵, (CH₂)ₖOC(O)R⁵, (CH₂)ₖNR⁵R⁶, or CN; or C₁₋₈ alkyl or cycloalkyl optionally substituted with one or more F, Cl, Br, I, OH, SH, ═O, ═S, NH₂, (CH₂)ₖCO₂R⁵, (CH₂)ₖNR⁵R⁶, OC₁₋₈ alkyl, or CN;
n is 0, 1, 2, 3, or 4;
each R² is, independently, F, Cl, Br, I, OH, SH, (CH₂)ₖCO₂R⁵, (CH₂)ₖOC(O)R⁵, (CH₂)ₖNR⁵R⁶, or CN; or C₁₋₈ alkyl or cycloalkyl optionally substituted with one or more F, Cl, Br, I, OH, SH, ═O, ═S, NH₂, (CH₂)ₖCO₂R⁵, (CH₂)ₖNR⁵R⁶, OC₁₋₈ alkyl, or CN;
m is 0, 1, 2, 3, or 4;
X is NH, NC₁₋₈ alkyl, NC(O)R⁵, CH₂, CH—C₁₋₈ alkyl, C(C₁₋₈ alkyl)₂, CO, O, S, SO, SO₂;
Y is N, CH;
R⁵ and R⁶ are, independently, H, C₁₋₈ alkyl, OC₁₋₈ alkyl, or cycloalkyl;
k is 0, 1, 2, 3, or 4;

(+), (−), and (±) isomers, and pharmaceutically acceptable salts and prodrugs thereof; wherein (a) is administered in an amount effective to enhance the therapeutic efficacy of (b) the anticancer drug. In some embodiments, (b) is administered in an amount of from 5% to 50% lower than an equivalent pharmaceutically effective amount administered without (a). In some embodiments, (b) is administered in an amount of from 5% to 50% lower than the recommended dosage of doxorubicin of 60 mg/m².

In some embodiments, (a) and (b) are administered simultaneously. In some embodiments, (a) and (b) are administered sequentially. In some embodiments, (a) and (b) are administered in a weight ratio of (a) to (b) from 1 to 1 to from 1 to 10.

In some embodiments, the method further comprises administering (c) a third compound or composition, wherein the third compound or composition includes an additional anti-cancer drug.

Additionally, the method can further comprise administering an effective amount of ionizing radiation to the subject. Methods of killing a tumor cell are also provided herein. The methods comprise contacting a tumor cell with an effective amount of one or more compounds or compositions disclosed herein.

Also provided herein are methods of radiotherapy of tumors, comprising contacting the tumor with an effective amount of one or more compounds or compositions as disclosed herein and irradiating the tumor with an effective amount of ionizing radiation.

Also disclosed are methods for treating oncological disorders in a patient. In one embodiment, an effective amount of one or more compounds or compositions disclosed herein is administered to a patient having an oncological disorder and who is in need of treatment thereof. The disclosed methods can optionally include identifying a subject who is or can be in need of treatment of an oncological disorder. The subject can be a human or other mammal, such as a primate (monkey, chimpanzee, ape, etc.), dog, cat, cow, pig, or horse, or other animals having an oncological disorder. Oncological disorders include, but are not limited to, cancer and/or tumors of the anus, bile duct, bladder, bone, bone marrow, bowel (including colon and rectum), breast, eye, gall bladder, kidney, mouth, larynx, esophagus, stomach, testis, cervix, head, neck, ovary, lung, mesothelioma, neuroendocrine, penis, skin, spinal cord, thyroid, vagina, vulva, uterus, liver, muscle, pancreas, prostate, blood cells (including lymphocytes and other immune system cells), and brain. In some embodiments, the cancer includes carcinomas, Karposi's sarcoma, melanoma, mesothelioma, soft tissue sarcoma, pancreatic cancer, lung cancer, leukemia (acute lymphoblastic, acute myeloid, chronic lymphocytic, chronic myeloid, and other), and lymphoma (Hodgkin's and non-Hodgkin's), multiple myeloma, prostate cancer, breast cancer, brain cancer, ovarian cancer, head and neck cancer, pancreatic cancer, cervical cancer, colon cancer, rectal cancer, endrometrial cancer, esophageal cancer, liver cancer, penile cancer, melanoma skin cancer, non-melanoma skin cancer, stomach cancer, testicular cancer, vaginal cancer, uterine cancer, vulvar cancer, paranasal cancer, oropharyngeal cancer, laryngeal cancer, or a combination thereof.

In specific examples, the disclosed topoisomerase II poisons (e.g., Compound 1) can be administered with doxorubicin to treat pediatric cancers, such as leukemias.

The compounds and/or compositions disclosed herein can, in some embodiments, shorten the yeast replicative lifespan at subtoxic concentrations (EC₅₀˜7 μM). The compounds and/or compositions disclosed herein can, in some embodiments, inhibit the activity of purified human topoisomerase II. The compounds and/or compositions disclosed herein can, in some embodiments, be nontoxic to both yeast and mammalian cells. Without wishing to be bound to theory, it is believed that compounds (a) can be nontoxic to yeast and mammalian cells because the broken double strand caused by the compounds is rapidly resolved by homologous recombination. Thus, the compounds (a) disclosed herein can be highly toxic in yeast cells deficient for homologous recombination.

It is also believed that, in some embodiments, compounds (a) can enhance the desirable effects and/or decrease the negative side effects of the anticancer drugs, compounds (b). Nontoxic doses of compounds (a), described above, can enhance the toxicity of compound (b) to cancer cells (e.g., human HT-1080 fibrosarcoma cells). Compound (a) can have no effect on the toxicity of compound (b) to a non-cancer human cell line. The compounds (a) disclosed herein can enhance other anti-cancer drugs that target topoisomerase II directly or indirectly.

### Administration

The term “administration” and variants thereof (e.g., “administering” a compound) in reference to a compound disclosed herein means introducing the compound, a salt or a prodrug thereof into the system of the animal in need of treatment. When a compound disclosed herein or prodrug thereof is provided in combination with one or more other active agents (e.g., a cytotoxic agent, etc.), “administration” and its variants are each understood to include concurrent and sequential introduction of the compound or prodrug thereof and other agents.

In vivo application of the disclosed compounds, and compositions containing them, can be accomplished by any suitable method and technique presently or prospectively known to those skilled in the art. For example, the disclosed compounds can be formulated in a physiologically acceptable form or pharmaceutically acceptable form and administered by any suitable route known in the art including, for example, oral, nasal, rectal, topical, and parenteral routes of administration. As used herein, the term parenteral includes subcutaneous, intradermal, intravenous, intramuscular, intraperitoneal, and intrasternal administration, such as by injection. Administration of the disclosed compounds or compositions can be a single administration, or at continuous or distinct intervals as can be readily determined by a person skilled in the art.

The compounds disclosed herein, and compositions comprising them, can also be administered using liposome technology, slow release capsules, implantable pumps, and biodegradable containers. These delivery methods can, advantageously, provide a uniform dosage over an extended period of time. The compounds can also be administered in their salt derivative forms or crystalline forms.

The compounds disclosed herein can be formulated according to known methods for preparing pharmaceutically acceptable compositions. Formulations are described in detail in a number of sources which are well known and readily available to those skilled in the art. For example, REMINGTON'S PHARMACEUTICAL SCIENCE by E. W. Martin (1995) describes formulations that can be used in connection with the disclosed methods. In general, the compounds disclosed herein can be formulated such that an effective amount of the compounds are combined with a suitable carrier to facilitate effective administration of the compounds. The compositions used can also be in a variety of forms. These include, for example, solid, semi-solid, and liquid dosage forms, such as tablets, pills, powders, liquid solutions or suspension, suppositories, injectable and infusible solutions, and sprays. The preferred form depends on the intended mode of administration and therapeutic application. The compositions can include conventional pharmaceutically acceptable carriers and diluents that are known to those skilled in the art. Examples of carriers or diluents for use with the compounds include ethanol, dimethyl sulfoxide, glycerol, alumina, starch, saline, and equivalent carriers and diluents. To provide for the administration of such dosages for the desired therapeutic treatment, compositions disclosed herein can advantageously comprise between about 0.1% and 99%, and especially, 1 and 15% by weight of the total of one or more of the subject compounds based on the weight of the total composition including carrier or diluent.

Formulations suitable for administration include, for example, aqueous sterile injection solutions, which can contain antioxidants, buffers, bacteriostats, and solutes that render the formulation isotonic with the blood of the intended recipient; and aqueous and nonaqueous sterile suspensions, which can include suspending agents and thickening agents. The formulations can be presented in unit-dose or multi-dose containers, for example sealed ampoules and vials, and can be stored in a freeze dried (lyophilized) condition requiring only the condition of the sterile liquid carrier, for example, water for injections, prior to use. Extemporaneous injection solutions and suspensions can be prepared from sterile powder, granules, tablets, etc. It should be understood that in addition to the ingredients particularly mentioned above, the compositions disclosed herein can include other agents conventional in the art having regard to the type of formulation in question.

Compounds disclosed herein, and compositions comprising them, can be delivered to a cell either through direct contact with the cell or via a carrier means. Carrier means for delivering compounds and compositions to cells are known in the art and include, for example, encapsulating the composition in a liposome moiety. Another means for delivery of compounds and compositions disclosed herein to a cell comprises attaching the compounds to a protein or nucleic acid that is targeted for delivery to the target cell. U.S. Pat. No. 6,960,648 and U.S. Application Publication Nos. 2003/0032594 and 2002/0120100 disclose amino acid sequences that can be coupled to another composition and that allows the composition to be translocated across biological membranes. U.S. Application Publication No. 2002/0035243 also describes compositions for transporting biological moieties across cell membranes for intracellular delivery. Compounds can also be incorporated into polymers, examples of which include poly (D-L lactide-co-glycolide) polymer for intracranial tumors; poly[bis(p-carboxyphenoxy) propane:sebacic acid] in a 20:80 molar ratio (as used in GLIADEL); chondroitin; chitin; and chitosan.

For the treatment of oncological disorders, the compounds disclosed herein can be administered to a patient in need of treatment in combination with other antitumor or anticancer substances and/or with radiation and/or photodynamic therapy and/or with surgical treatment to remove a tumor. These other substances or treatments can be given at the same as or at different times from the compounds disclosed herein. For example, the compounds disclosed herein can be used in combination with mitotic inhibitors such as taxol or vinblastine, alkylating agents such as cyclophosamide or ifosfamide, antimetabolites such as 5-fluorouracil or hydroxyurea, DNA intercalators such as adriamycin or bleomycin, topoisomerase inhibitors or poisons such as etoposide or camptothecin, antiangiogenic agents such as angiostatin, antiestrogens such as tamoxifen, and/or other anticancer drugs or antibodies, such as, for example, GLEEVEC (Novartis Pharmaceuticals Corporation) and HERCEPTIN (Genentech, Inc.), respectively.

In specific examples, the compounds disclosed herein can be administered to a patient in need of treatment in combination with doxorubicin, doxil (a pegylated liposomal formulation of doxorubicin), or Myocet (a nonpegylated liposomal formulation of doxorubicin. Other specific compounds that can be administered to a patient along with the compositions disclosed herein are ICRF-193, etoposide (VP-16), teniposide, daunorubicin, mitoxantrone, amsacrine, ellipticines, and aurintricarboxylic acid. That is compounds disclosed herein (e.g., compound 1) can be administered with, either in the same composition or formulation, simultaneously with, or before or after, doxorubicin, doxil, myocet, ICRF-193, etoposide (VP-16), teniposide, daunorubicin, mitoxantrone, amsacrine, ellipticines, or aurintricarboxylic acid.

Many tumors and cancers have viral genome present in the tumor or cancer cells. For example, Epstein-Barr Virus (EBV) is associated with a number of mammalian malignancies. The compounds disclosed herein can also be used alone or in combination with anticancer or antiviral agents, such as ganciclovir, azidothymidine (AZT), lamivudine (3TC), etc., to treat patients infected with a virus that can cause cellular transformation and/or to treat patients having a tumor or cancer that is associated with the presence of viral genome in the cells. The compounds disclosed herein can also be used in combination with viral based treatments of oncologic disease. For example, the compounds can be used with mutant herpes simplex virus in the treatment of non-small cell lung cancer (Toyoizumi et al., “Combined therapy with chemotherapeutic agents and herpes simplex virus type IICP34.5 mutant (HSV-1716) in human non-small cell lung cancer,” Human Gene Therapy, 1999, 10(18):17).

Therapeutic application of compounds and/or compositions containing them can be accomplished by any suitable therapeutic method and technique presently or prospectively known to those skilled in the art. Further, compounds and compositions disclosed herein have use as starting materials or intermediates for the preparation of other useful compounds and compositions.

Compounds and compositions disclosed herein can be locally administered at one or more anatomical sites, such as sites of unwanted cell growth (such as a tumor site or benign skin growth, e.g., injected or topically applied to the tumor or skin growth), optionally in combination with a pharmaceutically acceptable carrier such as an inert diluent. Compounds and compositions disclosed herein can be systemically administered, such as intravenously or orally, optionally in combination with a pharmaceutically acceptable carrier such as an inert diluent, or an assimilable edible carrier for oral delivery. They can be enclosed in hard or soft shell gelatin capsules, can be compressed into tablets, or can be incorporated directly with the food of the patient's diet. For oral therapeutic administration, the active compound can be combined with one or more excipients and used in the form of ingestible tablets, buccal tablets, troches, capsules, elixirs, suspensions, syrups, wafers, aerosol sprays, and the like.

The tablets, troches, pills, capsules, and the like can also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring can be added. When the unit dosage form is a capsule, it can contain, in addition to materials of the above type, a liquid carrier, such as a vegetable oil or a polyethylene glycol. Various other materials can be present as coatings or to otherwise modify the physical form of the solid unit dosage form. For instance, tablets, pills, or capsules can be coated with gelatin, wax, shellac, or sugar and the like. A syrup or elixir can contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active compound can be incorporated into sustained-release preparations and devices.

Compounds and compositions disclosed herein, including pharmaceutically acceptable salts, hydrates, or analogs thereof, can be administered intravenously, intramuscularly, or intraperitoneally by infusion or injection. Solutions of the active agent or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations can contain a preservative to prevent the growth of microorganisms.

The pharmaceutical dosage forms suitable for injection or infusion can include sterile aqueous solutions or dispersions or sterile powders comprising the active ingredient, which are adapted for the extemporaneous preparation of sterile injectable or infusible solutions or dispersions, optionally encapsulated in liposomes. The ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. Optionally, the prevention of the action of microorganisms can be brought about by various other antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the inclusion of agents that delay absorption, for example, aluminum monostearate and gelatin.

Sterile injectable solutions are prepared by incorporating a compound and/or agent disclosed herein in the required amount in the appropriate solvent with various other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

For topical administration, compounds and agents disclosed herein can be applied in as a liquid or solid. However, it will generally be desirable to administer them topically to the skin as compositions, in combination with a dermatologically acceptable carrier, which can be a solid or a liquid. Compounds and agents and compositions disclosed herein can be applied topically to a subject's skin to reduce the size (and can include complete removal) of malignant or benign growths, or to treat an infection site. Compounds and agents disclosed herein can be applied directly to the growth or infection site. The compounds and agents can be applied to the growth or infection site in a formulation such as an ointment, cream, lotion, solution, tincture, or the like. Drug delivery systems for delivery of pharmacological substances to dermal lesions can also be used, such as that described in U.S. Pat. No. 5,167,649.

Useful solid carriers include finely divided solids such as talc, clay, microcrystalline cellulose, silica, alumina and the like. Useful liquid carriers include water, alcohols or glycols or water-alcohol/glycol blends, in which the compounds can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers, for example.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user. Examples of useful dermatological compositions which can be used to deliver a compound to the skin are disclosed in U.S. Pat. No. 4,608,392; U.S. Pat. No. 4,992,478; U.S. Pat. No. 4,559,157; and U.S. Pat. No. 4,820,508.

Useful dosages of the compounds and agents and pharmaceutical compositions disclosed herein can be determined by comparing their in vitro activity, and in vivo activity in animal models. Methods for the extrapolation of effective dosages in mice, and other animals, to humans are known to the art; for example, see U.S. Pat. No. 4,938,949.

Also disclosed are pharmaceutical compositions that comprise a compound disclosed herein in combination with a pharmaceutically acceptable carrier. Pharmaceutical compositions adapted for oral, topical or parenteral administration, comprising an amount of a compound are disclosed. The dose administered to a patient, particularly a human, should be sufficient to achieve a therapeutic response in the patient over a reasonable time frame, without lethal toxicity, and preferably causing no more than an acceptable level of side effects or morbidity. One skilled in the art will recognize that dosage will depend upon a variety of factors including the condition (health) of the subject, the body weight of the subject, kind of concurrent treatment, if any, frequency of treatment, therapeutic ratio, as well as the severity and stage of the pathological condition.

For the treatment of oncological disorders, compounds and compositions disclosed herein can be administered to a patient in need of treatment prior to, subsequent to, or in combination with other antitumor or anticancer drugs or substances (e.g., chemotherapeutic agents, immunotherapeutic agents, radiotherapeutic agents, cytotoxic agents, etc.) and/or with radiation therapy and/or with surgical treatment to remove a tumor. For example, compounds and agents and compositions disclosed herein can be used in methods of treating cancer wherein the patient is to be treated or is or has been treated with mitotic inhibitors such as taxol or vinblastine, alkylating agents such as cyclophosamide or ifosfamide, antimetabolites such as 5-fluorouracil or hydroxyurea, DNA intercalators such as adriamycin or bleomycin, antiangiogenic agents such as angiostatin, antiestrogens such as tamoxifen, and/or other anti-cancer drugs or antibodies, such as, for example, GLEEVEC (Novartis Pharmaceuticals Corporation) and HERCEPTIN (Genentech, Inc.), respectively. These other substances or radiation treatments can be given at the same as or at different times from the compounds disclosed herein. Examples of other suitable chemotherapeutic agents include, but are not limited to, altretamine, bleomycin, bortezomib (VELCADE), busulphan, calcium folinate, capecitabine, carboplatin, carmustine, chlorambucil, cisplatin, cladribine, crisantaspase, cyclophosphamide, cytarabine, dacarbazine, docetaxel, epirubicin, fludarabine, fluorouracil, gefitinib (IRESSA), gemcitabine, hydroxyurea, idarubicin, ifosfamide, imatinib (GLEEVEC), irinotecan, liposomal doxorubicin, lomustine, melphalan, mercaptopurine, methotrexate, mitomycin, mitoxantrone, oxaliplatin, pentostatin, procarbazine, raltitrexed, streptozocin, tegafur-uracil, temozolomide, thiotepa, tioguanine/thioguanine, topotecan, treosulfan, vinblastine, vincristine, vindesine, vinorelbine. Examples of suitable immunotherapeutic agents include, but are not limited to, alemtuzumab, cetuximab (ERBITUX), gemtuzumab, iodine 131 tositumomab, rituximab, trastuzamab (HERCEPTIN). Cytotoxic agents include, for example, radioactive isotopes (e.g., I¹³¹, I¹²⁵, Y⁹⁰, P³², etc.), and toxins of bacterial, fungal, plant, or animal origin (e.g., ricin, botulinum toxin, anthrax toxin, aflatoxin, jellyfish venoms (e.g., box jellyfish), etc.) Also disclosed are methods for treating an oncological disorder comprising administering an effective amount of a compound and/or agent disclosed herein prior to, subsequent to, and/or in combination with administration of a chemotherapeutic agent, an immunotherapeutic agent, a radiotherapeutic agent, or radiotherapy.

### Kits

Kits for practicing the methods disclosed herein are further provided. By “kit” is intended any manufacture (e.g., a package or a container) comprising at least one compound or composition disclosed herein. The kit may be promoted, distributed, or sold as a unit for performing the methods of the present disclosure. Additionally, the kits may contain a package insert describing the kit and methods for its use. Any or all of the kit reagents may be provided within containers that protect them from the external environment, such as in sealed containers or pouches.

To provide for the administration of such dosages for the desired therapeutic treatment, in some embodiments, pharmaceutical compositions disclosed herein can comprise between about 0.1% and 45%, and especially, 1 and 15%, by weight of the total of one or more of the compounds based on the weight of the total composition including carrier or diluents. Illustratively, dosage levels of the administered active ingredients can be: intravenous, 0.01 to about 20 mg/kg; intraperitoneal, 0.01 to about 100 mg/kg; subcutaneous, 0.01 to about 100 mg/kg; intramuscular, 0.01 to about 100 mg/kg; orally 0.01 to about 200 mg/kg, e.g., about 1 to 100 mg/kg; intranasal instillation, 0.01 to about 20 mg/kg; and aerosol, 0.01 to about 20 mg/kg of animal (body) weight.

Also disclosed are kits that comprise a composition comprising a compound disclosed herein in one or more containers. The disclosed kits can optionally include pharmaceutically acceptable carriers and/or diluents. In one embodiment, a kit includes one or more other components, adjuncts, or adjuvants as described herein. In another embodiment, a kit includes one or more anti-cancer drugs, such as those agents described herein. In one embodiment, a kit includes instructions or packaging materials that describe how to administer a compound or composition of the kit. Containers of the kit can be of any suitable material, e.g., glass, plastic, metal, etc., and of any suitable size, shape, or configuration. In one embodiment, a compound and/or agent disclosed herein is provided in the kit as a solid, such as a tablet, pill, or powder form. In another embodiment, a compound and/or agent disclosed herein is provided in the kit as a liquid or solution. In one embodiment, the kit comprises an ampoule or syringe containing a compound and/or agent disclosed herein in liquid or solution form.

A number of embodiments of the disclosure have been described. Nevertheless, it will be understood that various modifications may be made without departing from the spirit and scope of the invention. Accordingly, other embodiments are within the scope of the following claims. By way of non-limiting illustration, examples of certain embodiments of the present disclosure are given below.

### EXAMPLES

The following examples are set forth below to illustrate the methods, compositions, and results according to the disclosed subject matter. These examples are not intended to be inclusive of all aspects of the subject matter disclosed herein, but rather to illustrate representative methods, compositions, and results. These examples are not intended to exclude equivalents and variations of the present invention, which are apparent to one skilled in the art.

### Example 1

### 6H-indolo[2,3-b]quinoxaline (Compound 1) inhibits human Top2-α at Micromolar Concentrations

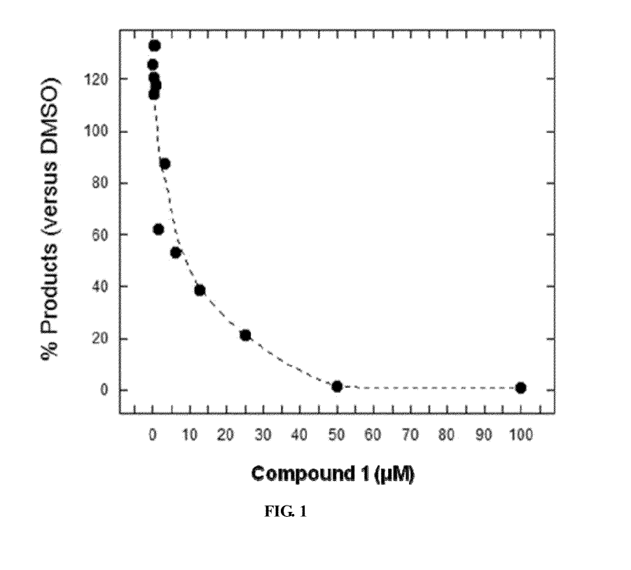

Human Top2-α decatenation of kinetoplastid DNA (kDNA) was visualized by gel electrophoresis. Human Top2-α (Topogen, Inc; Port Orange Fla.) was added to 0.2 μg of kDNA in the presence of 1% DMSO (vehicle control) without or with increasing concentrations of Compound 1. Reactions were incubated at 37° C. for 15 min and terminated by addition of an SDS buffer. Samples were resolved by 1% agarose 1×TAE gel electrophoresis and stained with ethidium bromide. Decatenated versus intact kDNA species were quantified by densitometry (FIG. 1).

### Example 2

### 6H-indolo[2,3-b]quinoxaline (Compound 1) is not a DNA Intercalating Agent

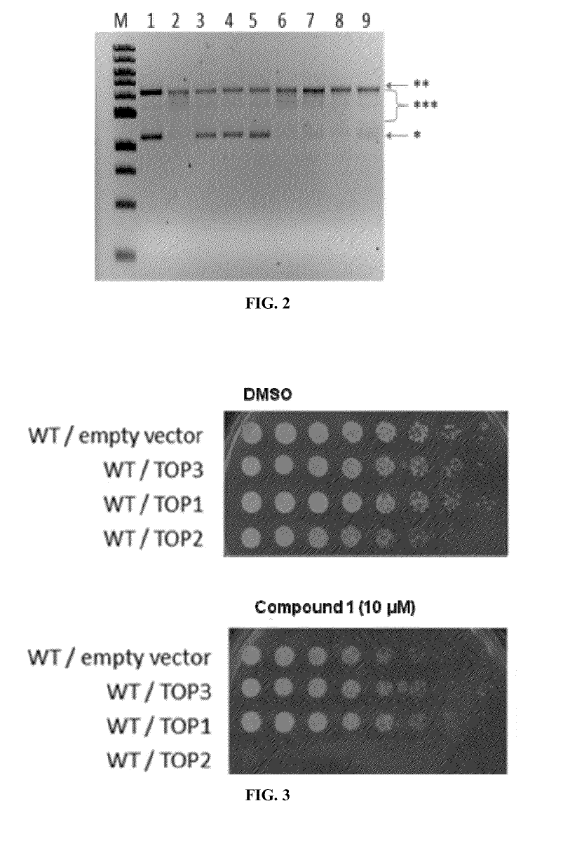

A Topoisomerase I (Top1) DNA unwinding assay was used to assess the ability of Compound 1 to intercalate. Doxorubicin and etoposide were included as control molecules that both inhibit Top2, but do or do not intercalate into DNA respectively. puc118-based plasmid DNA containing both supercoiled (*) and relaxed (**) forms was used as substrate and Top1 was purchased from Topogen, Inc. (Port Orange, Fla.). DNA was incubated with DMSO alone or DMSO containing the indicated amount of doxorubicin, etoposide, or Compound 1 for 15 min at 25° C., followed by an additional 1 h with added Top1. Samples were resolved by electrophoresis through 1% agarose 1×TAE gel and species *, **, and *** quantitated by densitometry (FIG. 2). Top1 alone (Lane 2) converts the DNA to a relaxed form (**) and some residual intermediate relaxed species (***). In the presence of the intercalator doxorubicin (lanes 3-5), DNA is unwound to a form that is indistinguishable from the parental supercoiled form (*). Non-intercalators such as etoposide (lane 6) and Compound 1 (lanes 8-9) do not unwind the DNA.

### Example 3

### 6H-indolo[2,3-b]quinoxaline (Compound 1) is Toxic to Yeast Cells Overexpressing TOP2

Three-fold dilutions of Saccharomyces cerevisiae strain BY4741 overexpressing yeast TOP1, TOP2, or TOP3 from plasmid vectors were plated on agar media containing either 0.1% DMSO or Compound 1 (10 μM) in 0.1% DMSO (FIG. 3). Growth was recorded after 48 h. TOP1, TOP2, and TOP3 overexpressing cells grew at approximately the same rate as cells carrying an empty vector (top panel). Compound 1 had no effect on the growth of cells carrying an empty vector or overexpressing TOP1 or TOP3. However, Compound 1 was toxic to cells overexpressing TOP2. This phenomenon is characteristic of TOP2 poisons that stabilize toxic Top2cc adducts.

### Example 4

### 6H-indolo[2,3-b]quinoxaline (Compound 1) Exhibits Limited Toxicity in Yeast Cells Expressing Native Levels of TOP2

Yeast cultures overexpressing native TOP2 (TOP2) or an empty expression vector (empty) were exposed to increasing concentrations of Compound 1 (FIG. 4). Even though Top2 is an essential protein in yeast, and other TOP2 poisons cause cell death, Compound 1 exhibits partial dose-dependent inhibition of growth rates in cells expressing native levels of Top2 at concentrations below 10 μM. At higher concentrations of Compound 1 growth rate inhibition does not increase, and cells continue to thrive at concentrations as high as 100 μM. Consistent with Compound 1 being a TOP2 poison, Compound 1 almost completely arrests growth in cells overexpressing TOP2 from a plasmid vector. These results indicate that cells are able to repair any DNA damage induced by Compound 1, at least at native levels of Top2. This is an unusual property for a TOP2 poison, most or all of which are lethal at high concentrations.

### Example 5

### 6H-indolo[2,3-b]quinoxaline (Compound 1) Induces Stable Double Strand Breaks in Plasmid DNA by Purified Yeast Top2

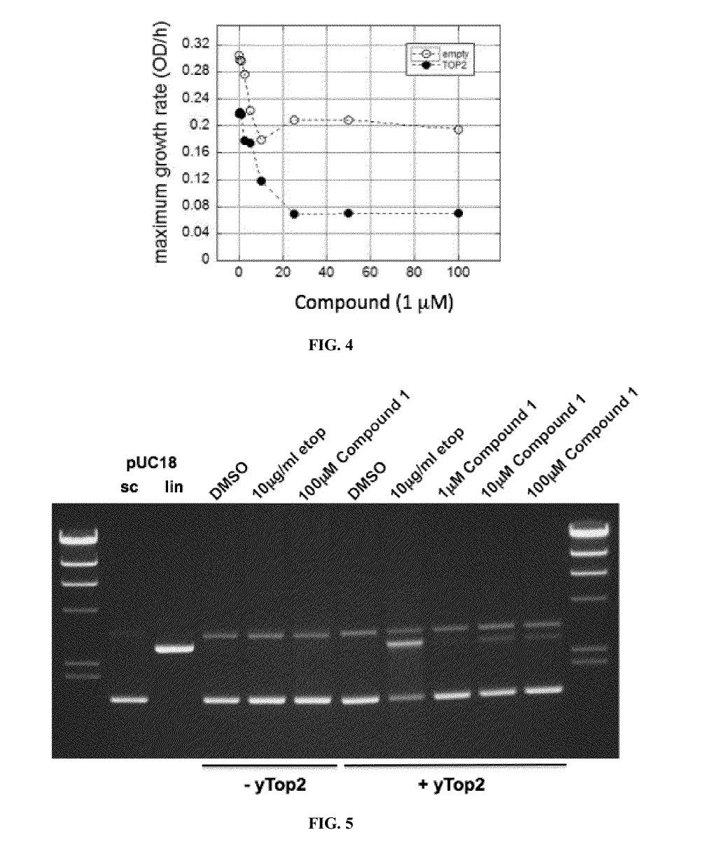

Supercoiled (sc) plasmid DNA (pUC18) was exposed to 10 μM etoposide or 1, 10 or 100 μM Compound 1 in the presence and absence of 150 ng of purified yeast Top2 as described (Nitiss et al. (2012) Curr Protoc. Pharmacol. Chapter 3: Unit 3.3) (FIG. 5). TOP2 poisons induce Top2cc adducts containing double strand breaks. Double strand DNA breaks convert supercoiled (sc) pUC18 to a linear (lin) form. Both etoposide, a well characterized TOP2 poison, and Compound 1 induced yeast Top2 to form the linear form of pUC18, consistent with the formation of Top2cc adducts.

### Example 6

### 6H-indolo[2,3-b]quinoxaline (Compound 1) Selectively Enhances Doxorubicin-Mediated Killing of Cancer Cells

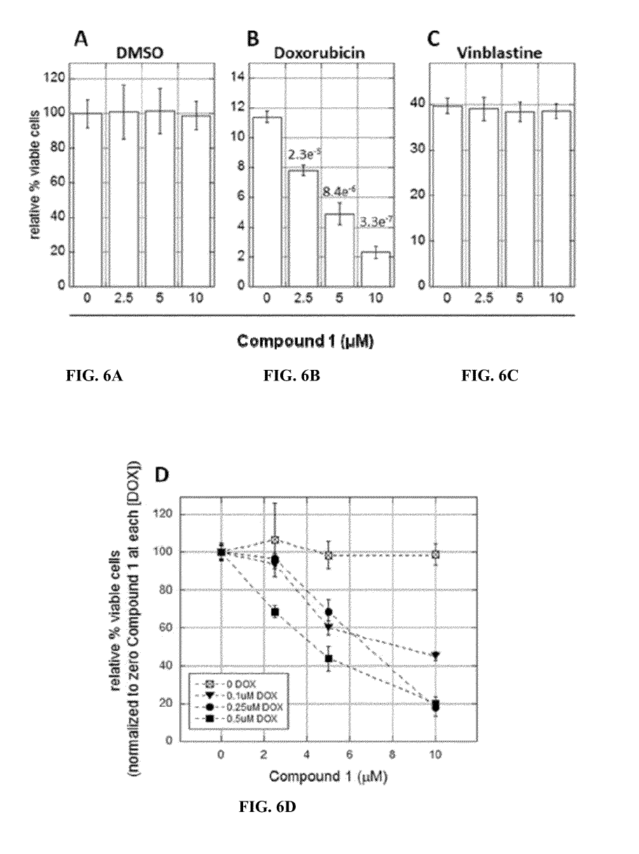

Human HT1080 fibrosarcoma cells were exposed for 48 hrs to DMSO (vehicle control), 0.5 μM doxorubicin, 0.5 nM vinblastine, and/or Compound 1 at the indicated concentrations (FIGS. 6A-6C, respectively). Viable cells remaining on the plates were quantified after 48 hrs. (FIG. 6A) Compound 1 has no effect on the viability of HT1080 cells at concentrations up to 10 nM. Thus Compound 1 is not itself toxic to cancer cells. (FIG. 6B) Compound 1 enhances doxorubicin toxicity at concentrations as low as 2.5 μM. 10 μM Compound 1 enhances doxorubicin cytotoxicity several fold. (FIG. 6C) Compound 1 does not enhance vinblastine toxicity. The vinblastine results are inconsistent with Compound 1 acting by inhibition of multidrug resistance pumps, which, when inhibited, increase the toxicity of multiple cytotoxic drugs including vinblastine. Compound 1 exhibits dose-dependent enhancement of HT1080 cell killing at multiple doxorubicin concentrations (FIG. 6D). These results demonstrate that Compound 1 exhibits the same dose-dependent enhancement activity at multiple doxorubicin concentrations. Average values and standard deviation of the means are plotted. P-values determined by student t-test are shown for doxorubicin enhancement (B). DMSO (A) and doxorubicin (B) data were based on the average of 6 biological replicates. Vinblastine data (C) was based on three biological replicates. Similar results in these cells were obtained with etoposide (data not shown), indicating that Compound 1 has a general activity of enhancing different classes of TOP2 poisons.

### Example 7

### 6H-indolo[2,3-b]quinoxaline (Compound 1) does not Enhance Doxorubicin Cytotoxicity to Noncancer MJT Cells (Primary Foreskin Fibroblasts)

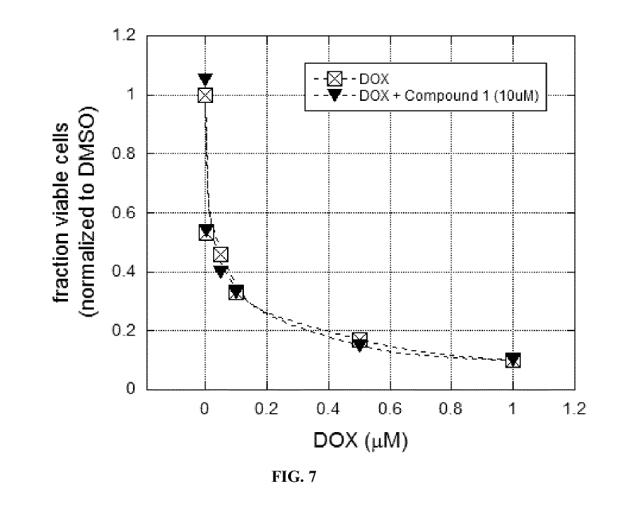

The results in FIG. 7 indicate that Compound 1 is not itself toxic to MJT cells, nor does it enhance the toxicity of doxorubicin in MJT cells. Thus Compound 1 exhibits selective toxicity in conjunction with doxorubicin towards cancer cells (see Example 6). This experiment was performed as described in Example 6.

Efforts have been made to ensure accuracy with respect to numbers (e.g., amounts, temperature, etc.) but some errors and deviations should be accounted for. Unless indicated otherwise, temperature is in ° C. or is at ambient temperature, and pressure is at or near atmospheric. There are numerous variations and combinations of reaction conditions, e.g., component concentrations, temperatures, pressures, and other reaction ranges and conditions that can be used to optimize the product purity and yield obtained from the described process. Only reasonable and routine experimentation will be required to optimize such process conditions.

The compositions and methods of the appended claims are not limited in scope by the specific compositions and methods described herein, which are intended as illustrations of a few aspects of the claims and any compositions and methods that are functionally equivalent are intended to fall within the scope of the claims. Various modifications of the compositions and methods in addition to those shown and described herein are intended to fall within the scope of the appended claims. Further, while only certain representative compositions and methods disclosed herein are specifically described, other combinations of the compositions and methods also are intended to fall within the scope of the appended claims, even if not specifically recited. Thus, a combination of steps, elements, components, or constituents may be explicitly mentioned herein; however, other combinations of steps, elements, components, and constituents are included, even though not explicitly stated.

It will be appreciated that variants of the above-disclosed and other features and functions, or alternatives thereof, may be combined into many other different systems or applications. Various presently unforeseen or unanticipated alternatives, modifications, variations, or improvements therein may be subsequently made by those skilled in the art which are also intended to be encompassed by the following claims.

What is claimed is:

1. A composition, comprising: a topoisomerase II poison of Formula I: 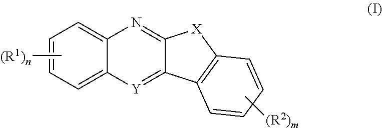
wherein
each R¹ is, independently, F, Cl, Br, I, OH, SH, (CH₂)ₖCO₂R⁵, (CH₂)ₖOC(O)R⁵, (CH₂)ₖNR⁵R⁶, or CN; or C₁₋₈ alkyl or cycloalkyl optionally substituted with one or more F, Cl, Br, I, OH, SH, ═O, ═S, NH₂, (CH₂)ₖCO₂R⁵, (CH₂)ₖNR⁵R⁶, OC₁₋₈ alkyl, or CN;
n is 0, 1, 2, 3, or 4;
each R² is, independently, F, Cl, Br, I, OH, SH, (CH₂)ₖCO₂R⁵, or (CH₂)ₖOC(O)R⁵, (CH₂)ₖNR⁵R⁶, or CN; or C₁₋₈ alkyl or cycloalkyl optionally substituted with one or more F, Cl, Br, I, OH, SH, ═O, ═S, NH₂, (CH₂)ₖCO₂R⁵, (CH₂)ₖNR⁵R⁶, OC₁₋₈ alkyl, or CN;
m is 0, 1, 2, 3, or 4;
X is N, NH, NC₁₋₈ alkyl, NC(O)R⁵, CH₂, CH—C₁₋₈ alkyl, C(C₁₋₈ alkyl)₂, CO, O, S, SO, or SO₂;
Y is N, NH, or CH;
R⁵ and R⁶ are, independently, H, C₁₋₈ alkyl, OC₁₋₈ alkyl, or cycloalkyl;
k is 0, 1, 2, 3, or 4;
a (+), (−), or (±) isomer thereof, or a pharmaceutically acceptable salt or prodrug thereof.

2. The composition of claim 1, wherein the topoisomerase II poison is 6H-indolo[2,3-b]quinoxaline or 11H-indeno[1,2-b]quinoxalin-11-one.

3. The composition of claim 1, wherein Y is N.

4. The composition of claim 1, wherein X is NH or CO.

5. The composition of claim 1, wherein n is 0.

6. The composition of claim 1, wherein m is 0.

7. The composition of claim 1, further comprising an anticancer drug.

8. The composition of claim 7, wherein the anticancer drug comprises a polycyclic chromophore, a synthetic intercalating drug, an antitumor antibiotic, a fluoroquinolone, a combination thereof, a (+), (−), or (±) isomer thereof, or a pharmaceutically acceptable salt or prodrug thereof.

9. The composition of claim 7, wherein the anticancer drug comprises a polycyclic chromophore, which comprises a benzoisoquinolinedione, an anthrapyrazole, a phenazine-1-carboxamide, a combination thereof, a (+), (−), or (±) isomer thereof, or a pharmaceutically acceptable salt or prodrug thereof.

10. The composition of claim 7, wherein the anticancer drug comprises a synthetic intercalating drug, which comprises an aminoacridine, a (+), (−), or (±) isomer thereof, or a pharmaceutically acceptable salt or prodrug thereof.

11. The composition of claim 7, wherein the anticancer drug comprises an antitumor antibiotic, which comprises an anthracyline, a (+), (−), or (±) isomer thereof, or a pharmaceutically acceptable salt or prodrug thereof.

12. The composition of claim 7, wherein the anticancer drug is aclarubicin, actinomycin, amsacrine, bleomycin, camptothecin, ciprofloxacin, dactinomycin, daunorubicin, ellipticine, epipodophyllotoxin, etoposide, genistein, idarubicin, losoxantrone, merbarone, mitonafide, mitoxanthrone, paclitaxel, plicamycin, phosphate, teniposide, a combination thereof, a (+), (−), or (±) isomer thereof, or a pharmaceutically acceptable salt or prodrug thereof.

13. The composition of claim 7, wherein the anticancer drug is doxorubicin, a (+), (−), or (±) isomer thereof, or a pharmaceutically acceptable salt or prodrug thereof.

14. The composition of claim 7, wherein the anticancer drug is acridine-4-carboxamide N-[2-(dimethylamino)ethyl]acridine-4carboxamide, imidazoacridanone, a combination thereof, a (+), (−), or (±) isomer thereof, or a pharmaceutically acceptable salt or prodrug thereof.

15. The composition of claim 7, wherein the weight ratio of topoisomerase II poison to anticancer drug is from 1 to 1 to from 1 to 10.

16. A composition, comprising: (a) 6H-indolo[2,3-b]quinoxaline, 11H-indeno[1,2-b]quinoxalin-11-one, or a combination thereof, and (b) doxorubicin, doxil, myocet, or a combination thereof.

17. A method of treating cancer in a subject, comprising:
administering to the subject a pharmaceutically effective amount of (a) a topoisomerase II poison of Formula I 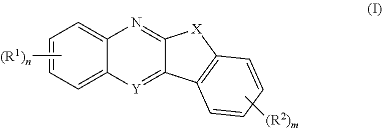
wherein
each R¹ is, independently, F, Cl, Br, I, OH, SH, (CH₂)ₖCO₂R⁵, (CH₂)ₖOC(O)R⁵, (CH₂)ₖNR⁵R⁶, or CN; or C₁₋₈ alkyl or cycloalkyl optionally substituted with one or more F, Cl, Br, I, OH, SH, ═O, ═S, NH₂, (CH₂)ₖCO₂R⁵, (CH₂)ₖNR⁵R⁶, OC₁₋₈ alkyl, or CN;
n is 0, 1, 2, 3, or 4;
each R² is, independently, F, Cl, Br, I, OH, SH, (CH₂)ₖCO₂R⁵, (CH₂)ₖOC(O)R⁵, (CH₂)ₖNR⁵R⁶, or CN; or C₁₋₈ alkyl or cycloalkyl optionally substituted with one or more F, Cl, Br, I, OH, SH, ═O, ═S, NH₂, (CH₂)ₖCO₂R⁵, (CH₂)ₖNR⁵R⁶, OC₁₋₈ alkyl, or CN;
m is 0, 1, 2, 3, or 4;
X is NH, NC₁₋₈ alkyl, NC(O)R⁵, CH₂, CH—C₁₋₈ alkyl, C(C₁₋₈ alkyl)₂, CO, O, S, SO, SO₂;
Y is N, CH;
R⁵ and R⁶ are, independently, H, C₁₋₈ alkyl, OC₁₋₈ alkyl, or cycloalkyl;
k is 0, 1, 2, 3, or 4;
a (+), (−), or (±) isomer thereof, or a pharmaceutically acceptable salt or prodrug thereof; and
(b) an anticancer drug.

18. The method of claim 17, wherein the topoisomerase II poison and anticancer drug are administered sequentially.

19. The method of claim 17, wherein the anticancer drug is administered in an amount of from 5% to 50% lower than an equivalent pharmaceutically effective amount administered without the topoisomerase II poison.

20. The method of claim 17, wherein the anticancer drug is doxorubicin and it is administered in an amount of from 5% to 50% lower than the recommended dose of doxorubicin of 60 mg/m².

21. The method of claim 17, wherein the cancer is prostate cancer, lung cancer, breast cancer, brain cancer, ovarian cancer, lymphoma, leukemia, head and neck cancer, pancreatic cancer, cervical cancer, colon cancer, rectal cancer, endrometrial cancer, esophageal cancer, liver cancer, penile cancer, melanoma skin cancer, non-melanoma skin cancer, stomach cancer, testicular cancer, vaginal cancer, uterine cancer, vulvar cancer, paranasal cancer, oropharyngeal cancer, laryngeal cancer, or a combination thereof.
